(19) United States
(12) Patent Application Publication
(10) Pub. No.: US 2023/0365564 A1
(43) Pub. Date: Nov. 16, 2023

(54) COCRYSTALS OF AN ADENOSINE A2B RECEPTOR ANTAGONIST

(71) Applicant: Teon Therapeutics, Inc., Redwood City, CA (US)

(72) Inventors: Jiwen LIU, Redwood City, CA (US); Drazen OSTOVIC, Redwood City, CA (US); Sami KARABORNI, Redwood City, CA (US)

(21) Appl. No.: 18/024,721

(22) PCT Filed: Sep. 3, 2021
(86) PCT No.: PCT/US2021/049011

§ 371 Date: Mar. 3, 2023

Related U.S. Application Data
(60) Provisional application No. 63/075,065, filed on Sep. 4, 2020.

(51) Int. Cl. C07D 473/06

(52) U.S. Cl. CPC C07D 473/06; C07B 2200/13

(57) ABSTRACT

Cocrystals of an adenosine A_{2B} receptor antagonist, 3-ethyl-1-propyl-8-(1-(3-(trifluoromethyl)benzyl)-1H-pyrazol-4-yl)-3,7-dihydro-1H-purine-2,6-dione, methods of preparing the cocrystals, pharmaceutical compositions of the cocrystals, and uses of the cocrystals are disclosed.

### CROSS REFERENCE TO RELATED APPLICATIONS

This application claims the benefit of priority under 35 U.S.C. § 119(e) to U.S. Provisional Application No. 63/075,065 filed Sep. 4, 2020, which is incorporated herein by reference in its entirety.

### FIELD

The disclosure relates to cocrystals of an adenosine A_{2B} receptor antagonist, 3-ethyl-1-propyl-8-(1-(3-(trifluoromethyl)benzyl)-1H-pyrazol-4-yl)-3,7-dihydro-1H-purine-2,6-dione, methods of preparing the cocrystals, pharmaceutical compositions containing the cocrystals, and uses of the cocrystals.

### BACKGROUND

For orally administered active pharmaceutical ingredients, the aqueous dissolution and membrane permeation process can be rate determining steps.

Increasing the solubility of a drug in gastrointestinal fluids can increase the oral bioavailability of the drug.

A pharmaceutical cocrystal is a crystalline form of a combination of an active pharmaceutical ingredient and a cocrystal former in a fixed stoichiometric ratio by weak interaction in which the active pharmaceutical ingredient can be in free form or be in a salt form.

A cocrystal can improve the physico-chemical properties of an active pharmaceutical ingredient.

### SUMMARY

According to the present disclosure, cocrystals comprise 3-ethyl-1-propyl-8-(1-(3-(trifluoromethyl)benzyl)-1H-pyrazol-4-yl)-3,7-dihydro-1H-purine-2,6-dione and benzene sulfonic acid, para-toluene sulfonic acid, or fumaric acid.

According to the present disclosure, methods of preparing a cocrystal, comprise combining 3-ethyl-1-propyl-8-(1-(3-(trifluoromethyl)benzyl)-1H-pyrazol-4-yl)-3,7-dihydro-1H-purine-2,6-dione and an organic acid in a non-polar solvent to form a suspension, wherein the organic acid is selected from benzenesulfonic acid, para-toluenesulfonic acid, and fumaric acid; heating the suspension; and cooling the heated suspension to a temperature from 20° C. to 30° C. and stirring the cooled suspension; to provide the corresponding cocrystal.

According to the present disclosure, pharmaceutical compositions comprise a cocrystal according to the present disclosure.

According to the present disclosure, methods of treating a disease in a patient comprise administering to a patient in need of such treatment a therapeutically effective amount of a cocrystal according to the present disclosure.

According to the present disclosure, methods of treating a disease in a patient comprise administering to a patient in need of such treatment a therapeutically effective amount of a pharmaceutical composition according to the present disclosure.

### BRIEF DESCRIPTION OF THE DRAWINGS

The drawings described herein are for illustration purposes only. The drawings are not intended to limit the scope of the present disclosure.

FIG. 1 shows an X-ray powder diffraction (XRPD) pattern of Compound (1).

FIG. 2 shows a differential scanning calorimetry (DSC) scan of Compound (1).

FIG. 3 shows a thermogravimetric analysis (TGA) scan of Compound (1).

FIG. 4 shows an XRPD pattern of a benzenesulfonic acid cocrystal provided by the present disclosure.

FIG. 5 shows an DSC scan of a benzenesulfonic acid cocrystal provided by the present disclosure.

FIG. 6 shows an TGA scan of a benzenesulfonic acid cocrystal provided by the present disclosure.

FIG. 7 shows an polarized light microscopy (PLM) image of benzenesulfonic acid cocrystals provided by the present disclosure.

FIG. 8 shows a scanning electron microscopy (SEM) image of benzenesulfonic acid cocrystals provided by the present disclosure.

FIG. 9 shows an XRPD pattern of a para-toluenesulfonic acid cocrystal provided by the present disclosure.

FIG. 10 shows an DSC scan of a para-toluenesulfonic acid cocrystal provided by the present disclosure.

FIG. 11 shows an TGA scan of a para-toluenesulfonic acid cocrystal provided by the present disclosure.

FIG. 12 shows an PLM image of para-toluenesulfonic acid cocrystals provided by the present disclosure.

FIG. 13 shows a SEM image of para-toluenesulfonic acid cocrystals provided by the present disclosure.

FIG. 14 shows an XRPD pattern of a fumaric acid cocrystal provided by the present disclosure.

FIG. 15 shows an DSC scan of a fumaric acid cocrystal provided by the present disclosure.

FIG. 16 shows an TGA scan of a fumaric cocrystal provided by the present disclosure.

FIG. 17 shows an polarized light microscopy image of fumaric acid cocrystals provided by the present disclosure.

FIG. 18 shows a scanning electron microscopy image of fumaric acid cocrystals provided by the present disclosure.

FIGS. 19-23 show XRPD patterns of cocrystals provided by the present disclosure after standing in a relevant biological fluid at 37° C. for 24 hours.

FIGS. 24-26 show kinetic solubility profiles for Compound (1) and the cocrystals provided by the present disclosure in (1) fed state simulated intestinal fluid version 1 (FaSSIF)/4% Soluplus®, (2) simulated gastric fluid (SGF)/4% Soluplus®, or (3) SGF, respectively.

FIGS. 27 and 28 show pharmacokinetic profiles for the fumaric acid cocrystal provided by the present disclosure following peroral administration to beagle dogs at a dose of 2 mg/kg or 10 mg/kg, respectively.

### DETAILED DESCRIPTION

For purposes of the following detailed description, it is to be understood that embodiments provided by the present disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

“Immediate release” refers to a pharmaceutical composition that releases substantially all of an pharmaceutically active ingredient into the gastrointestinal tract of a patient within less than 1 hour following oral administration, such as within less than 50 minutes, within less than 40 minutes, within less than 30 minutes, within less than 20 minutes, or within less than 10 minutes following oral administration. For example, an immediate release dosage form can release greater than 90%, greater than 95%, or greater than 98% of the pharmaceutically active ingredient in the pharmaceutical composition into the gastrointestinal tract within less than 1 hour such as within less than 50 minutes, less than 40 minutes, less than 30 minutes, less than 20 minutes, or less than 10 minutes, following oral administration. Immediate release pharmaceutical compositions can be appropriate to administer pharmaceutically active ingredients that are absorbed into the systemic circulation from the upper portion of the gastrointestinal tract.

“Modified release” pharmaceutical compositions include controlled release formulations, delayed release formulations, extended release formulations, sustained release formulations, timed release formulations, pulsatile release formulations, and pH-dependent release formulations. These formulations are intended to release a pharmaceutically active ingredient from the pharmaceutical composition at a desired rate and/or at a desired time following oral administration by a patient and/or at a certain location or locations within the gastrointestinal tract and/or at a certain pH within the gastrointestinal tract. The USP defines a modified release system as one in which the time course or location of drug release or both, are chosen to accomplish objectives of therapeutic effectiveness or convenience not fulfilled by immediate release dosage forms. A modified release oral dosage form can include extended release and delayed-release components. A delayed release dosage form is one that releases a drug all at once at a time other than promptly after administration. A modified release formulation can include delayed-release using enteric coatings, site-specific or timed release such as for colonic delivery, extended-release including, for example, formulations capable of providing zero-order, first-order, or biphasic release profiles, and programmed release such as pulsatile and delayed extended release.

“Sustained release” pharmaceutical compositions and coating provide for a dissolution rate over an extended period of time following oral administration. Granulations comprising granules having a sustained release coating can be referred to as sustained release granulations. A pharmaceutical composition comprising a sustained release granulation can be referred to as a sustained release pharmaceutical composition.

“pH-release” pharmaceutical compositions and coatings provide for an increased dissolution rate at an intended pH.

“Pulsatile release” pharmaceutical compositions and coatings exhibit an increased dissolution rate at intervals, where the release intervals can be determined by time, exposure to internal stimuli, or exposure to external stimuli. Examples of pulsatile-release systems include capsular systems, osmotic systems, systems having erodible membranes, and systems having a rupturable coating. Examples of stimuli include temperature, chemicals, electrical stimuli, and magnetic stimuli.

“Timed-release” pharmaceutical compositions and coatings have a dissolution rate that is a function of time. A time-release pharmaceutical composition or coating includes, for example, delayed release, sustained release, and extended release pharmaceutical compositions and coatings.

“Delayed release” pharmaceutical compositions and coatings provide for an increased dissolution rate at an intended time after administration.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Curing” a disease refers to eliminating a disease or disorder or eliminating a symptom of a disease or disorder.

“Treating” or “treatment” of a disease or disorder refers to reducing the severity of one or more clinical symptom of the disease or disorder, delaying the onset of one or more clinical symptoms of the disease or disorder, and/or mitigating one or more clinical symptoms of the disease or disorder.

“Treating” or “treatment” of a disease or disorder refers to inhibiting the disease or disorder or one or more clinical symptoms of the disease or disorder, arresting the development of the disease or disorder or one or more clinical symptoms of the disease or disorder, relieving the disease or disorder or one or more clinical symptoms of the disease or disorder, causing the regression of the disease or disorder or one or more clinical symptoms of the disease or disorder, and/or stabilization of the disease or disorder or one or more clinical symptoms of the disease or disorder. “Treating” or “treatment” of a disease or disorder refers to producing a clinically beneficial effect without curing the underlying disease or disorder.

“Therapeutically effective amount” refers to the amount of a compound or cocrystal, such as a pharmaceutically active ingredient that, when administered to a patient for treating a disease, or at least one of the clinical symptoms of a disease, is sufficient to affect such treatment of the disease or symptom thereof. A “therapeutically effective amount” may vary depending, for example, on the compound, the disease and/or symptoms of the disease, the severity of the disease and/or symptoms of the disease or disorder, the age, weight, and/or health of the patient to be treated, and the judgment of the prescribing physician. A therapeutically effective amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Vehicle” refers to a diluent, excipient or carrier with which a compound is administered to a patient. A vehicle can be a pharmaceutically acceptable vehicle. Pharmaceutically acceptable vehicles are known in the art.

Reference is now made to cocrystals, methods of making the cocrystals, pharmaceutical compositions comprising the cocrystals, and uses of the cocrystals. The disclosed cocrystals, pharmaceutical compositions, methods, and uses are not intended to be limiting of the claims. To the contrary, the claims are intended to cover all alternatives, modifications, and equivalents.

Reference is now made in detail to certain embodiments of compounds, compositions, and methods. The disclosed embodiments are not intended to be limiting of the claims. To the contrary, the claims are intended to cover all alternatives, modifications, and equivalents.

3-Ethyl-1-propyl-8-(1-(3-(trifluoromethyl)benzyl)-1H-pyrazol-4-yl)-3,7-dihydro-1H-purine-2,6-dione is an adenosine A_{2B} receptor antagonist that is being developed for the treatment of diseases such as asthma and cancer treatment. The compound has poor aqueous solubility which can lead to sub-optimal pharmacokinetics.

Cocrystals of 3-ethyl-1-propyl-8-(1-(3-(trifluoromethyl)benzyl)-1H-pyrazol-4-yl)-3,7-dihydro-1H-purine-2,6-dione and benzenesulfonic acid, para-toluenesulfonic acid, and fumaric acid have a high aqueous solubility and are stable in clinically relevant media.

3 -Ethyl-1-propyl-8-(1-(3-(trifluoromethyl)benzyl)-1H-pyrazol-4-yl)-3,7-dihydro-1H-purine-2,6-dione (compound (1)) has the structure of Formula (1):

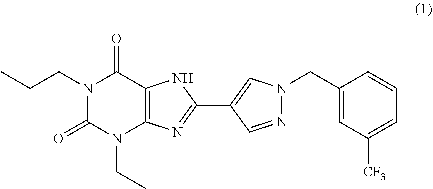 [SMILES: CCCn1c(=O)c2[nH]c(-c3cnn(Cc4cccc(C(F)(F)F)c4)c3)nc2n(CC)c1=O]

Methods of synthesizing compound (1) and properties of compound (1) are provided in U.S. Application Publication No. 2004/0176399 and in International Publication No. WO 2019/1733380.

Compound (1) has an aqueous solubility of less than 1 μg/mL that is substantially independent of pH over a relevant physiological range.

Compound (1) is an adenosine A_{2B} receptor antagonist.

Compound (1) can be characterized by an XRPD pattern comprising characteristic diffraction peaks at least at 6.8°±0.2°, 10.3°±0.2°, 13.6°±0.2°, 17.1±0.2°, and 21.4±0.2° expressed as 2θ angles and determined using Cu-Kα radiation.

Compound (1) can be characterized by an XRPD pattern comprising characteristic diffraction peaks at least at 6.8°±0.1°, 10.3°35 0.1°, 13.6°±0.1°, 17.1±0.1°, and 21.4±0.1° expressed as 2θ angles and determined using Cu-Kα radiation.

Compound (1) can be characterized by an XRPD pattern comprising characteristic diffraction peaks at least at 6.8°±0.2°, 10.3°±0.2°, 11.6, 13.6°±0.2°, 15.4±0.2°, 17.1±0.2°, 21.4±0.2°, 22.3±0.2°, 24.8±0.2°, and 27.4±0.2° expressed as 2θ angles and determined using Cu-Kα radiation.

Compound (1) can be characterized by an XRPD pattern comprising characteristic diffraction peaks at least at 6.8°±0.1°, 10.3°±0.1°, 11.6, 13.6°±0.1°, 15.4±0.1°, 17.1±0.1°, 21.4±0.1°, 22.3±0.1°, 24.8±0.1°, and 27.4±0.1° expressed as 2θ angles and determined using Cu-Kα radiation.

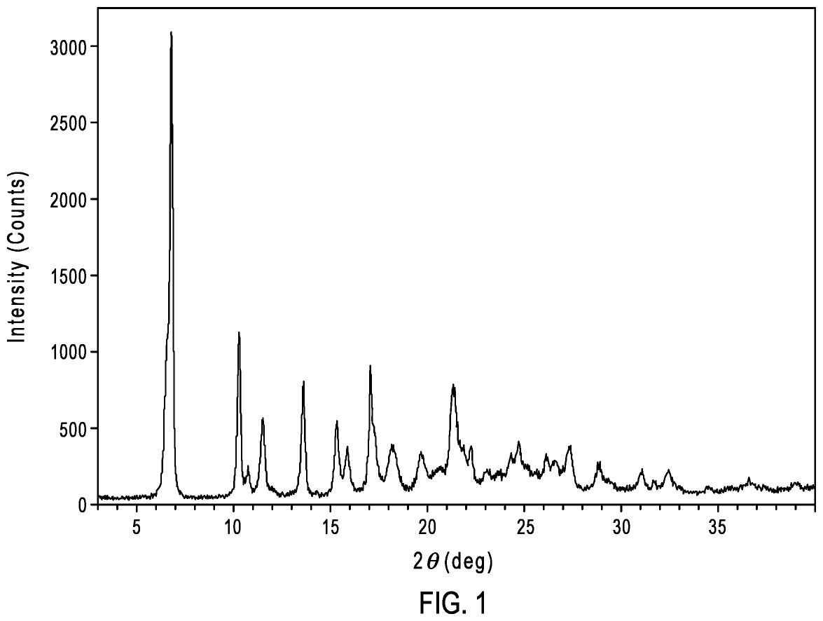

Compound (1) can be characterized by an XRPD pattern as substantially shown in FIG. 1.

Compound (1) can have a melting onset temperature, for example, from 254° C. to 260° C., such as from 255° C. to 259° C., or from 256° C. to 258° C., where the melting onset temperature is determined by differential scanning calorimetry.

Compound (1) can have a melting onset temperature, for example, of 257.2° C.±0.5° C., such as 257.2° C.±0.25° C., or 257.2° C.±0.1° C., where the melting onset temperature is determined by differential scanning calorimetry.

Compound (1) can have a melting enthalpy, for example, from 91 J/g to 101 J/g, from 93 J/g to 99 J/g, or from 95 J/g to 97 J/g, where the melting enthalpy is determined by differential scanning calorimetry.

Compound (1) can have a melting enthalpy, for example, of 96.0 J/g±0.5 J/g, such as 96.0 J/g±0.25 J/g, or 96.0 J/g±0.1 J/g, where the melting enthalpy is determined by differential scanning calorimetry.

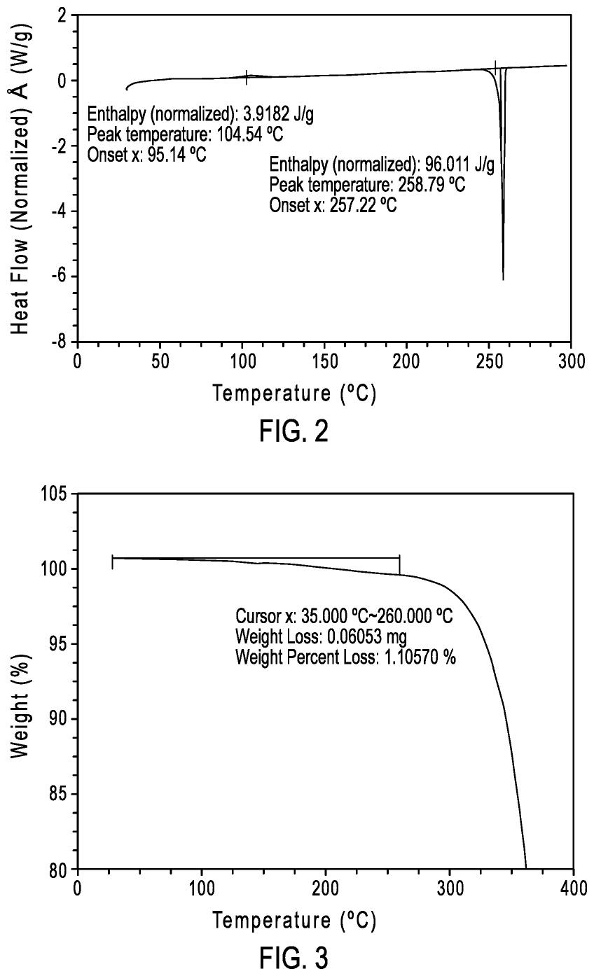

Compound (1) can exhibit a differential scanning calorimetry curve as substantially shown in FIG. 2.

Compound (1) can have a weight loss, for example, from 0.9% to 1.3% at a temperature from 255° C. to 265° C., such as from 0.95% to 1.25% at a temperature from 257° C. to 262° C., or from 1.0% to 1.2% at a temperature from 259° C. to 261° C., where the weight loss is determined by thermogravimetric analysis.

Compound (1) can have a weight loss, for example, of 1.1%±0.05% at 260° C.±5° C., such as 1.1%±0.02% at 260° C.±2.5° C., or 1.1%±0.01% at 260° C.±1° C., where the weight loss is determined by thermogravimetric analysis.

Compound (1) provided by the present disclosure can exhibit a differential thermal calorimetry curve as substantially shown in FIG. 3.

Compound (1) is stable, for example, in an open vial at 25° C./60% RH for 2 weeks, in an open vial at 40° C./75% RH for 2 weeks, and in a closed vial at 60° C. or 1.2M lux-hours for 2 weeks.

Cocrystals of compound (1) provided by the present disclosure include a benzenesulfonic acid cocrystal of compound (1), a para-toluenesulfonic acid cocrystal of compound (1), and a fumaric acid cocrystal of compound (1).

A benzenesulfonic acid cocrystal of compound (1) can comprise, for example, from 1.0 equivalents to 1.4 equivalents, such as from 1.05 equivalents to 1.35 equivalents, from 1.1 equivalents to 1.3 equivalents, or from 1.15 equivalents to 1.25 equivalents benzenesulfonic acid, wherein the stoichiometry is determined by ¹H-NMR.

A benzenesulfonic acid cocrystal of compound (1) can comprise, for example, 1.2 equivalents of benzenesulfonic acid.

A benzenesulfonic acid cocrystal of compound (1) provided by the present disclosure can be characterized by an XRPD pattern comprising characteristic diffraction peaks and determined using Cu-Kα radiation.

A benzenesulfonic acid cocrystal of Compound (1) provided by the present disclosure can be characterized by an XRPD pattern comprising characteristic diffraction peaks at 6.8°±0.1°, 17.2°±0.1°, and 23.6°±0.1° expressed as 2θ angles and determined using Cu-Kα radiation.

A benzenesulfonic acid cocrystal of compound (1) provided by the present disclosure can be characterized by an XRPD pattern comprising characteristic diffraction peaks at 6.9°±0.2°, 14.7°±0.2°, 15.6°±0.2°, 17.2°±0.2°, 18.8°±0.2°, 22.3°±0.2°, and 23.6°±0.2° expressed as 2θ angles and determined using Cu-Kα radiation.

A benzenesulfonic acid cocrystal of compound (1) provided by the present disclosure can be characterized by an XRPD pattern comprising characteristic diffraction peaks at 6.9°±0.1°, 14.7°±0.1°, 15.6°±0.1°, 17.2°, 18.8°±0.1°, 22.3°±0.1°, and 23.6°±0.1° expressed as 2θ angles and determined using Cu-Kα radiation.

A benzenesulfonic acid cocrystal of compound (1) provided by the present disclosure can be characterized by an XRPD pattern comprising characteristic diffraction peaks at 6.9°±0.2°, 9.9°±0.2°, 14.7°±0.2°, 15.6°±0.2°, 17.2°±0.2°, 18.8°±0.2°, 20.4°±0.2°, 22.3°±0.2°, 23.6°±0.2°, 25.0°±0.2°, 26.0°±0.2°, 27.2°±0.2°, 28.66°±0.2°, and 31.4°±0.2° expressed as 2θ angles and determined using Cu-Kα radiation.

A benzenesulfonic acid cocrystal of compound (1) provided by the present disclosure can be characterized by an XRPD pattern comprising characteristic diffraction peaks at 6.9°±0.1°, 9.9°±0.1°, 14.7°±0.1°, 15.6°±0.1°, 17.2°±0.1°, 18.8°±0.1°, 20.4°±0.1°, 22.3°±0.1°, 23.6°±0.1°, 25.0°±0.1°, 26.0°±0.1°, 27.2°±0.1°, 28.66°±0.1°, and 31.4°±0.1° expressed as 2θ angles and determined using Cu-Kα radiation.

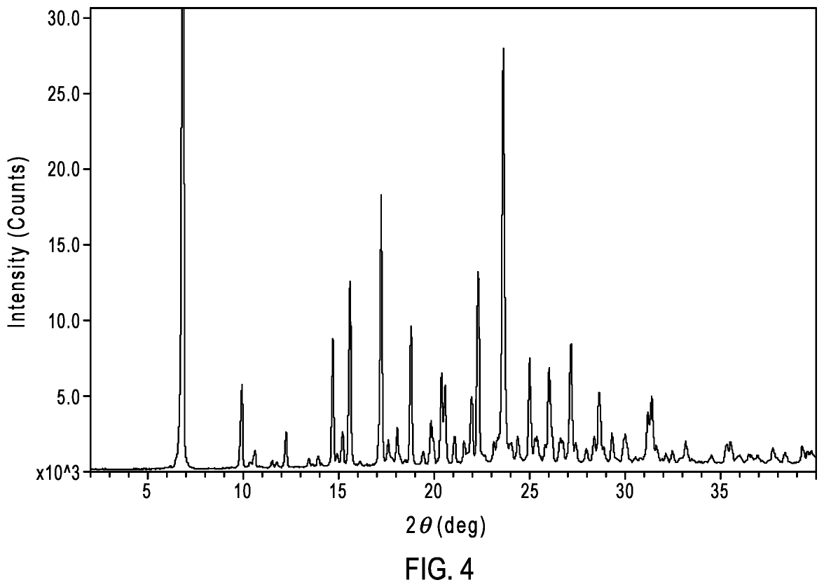

A benzenesulfonic acid cocrystal of compound (1) provided by the present disclosure can be characterized by an XRPD pattern as substantially shown in FIG. 4.

A benzenesulfonic acid cocrystal of compound (1) provided by the present disclosure can have a melting onset temperature, for example, from 161° C. to 171° C., such as from 161° C. to 170° C., from 162° C. to 169° C., from 163° C. to 168° C., or from 164° C. to 167° C., where the melting onset temperature is determined by differential scanning calorimetry.

A benzenesulfonic acid cocrystal of compound (1) provided by the present disclosure can have a melting onset temperature, for example, of 166.3° C.±0.5° C., such as 166.3° C.±0.25° C., or 166.3° C.±0.1° C., where the melting onset temperature is determined by differential scanning calorimetry.

A benzenesulfonic acid cocrystal of compound (1) provided by the present disclosure can have a melting enthalpy, for example, from 65 J/g to 75 J/g, from 66 J/g to 74 J/g, from 67 J/g to 73 J/g, from 68 J/g to 72, or from 69 J/g to 71 J/g, where the melting enthalpy is determined by differential scanning calorimetry.

A benzenesulfonic acid cocrystal of compound (1) provided by the present disclosure can have a melting enthalpy, for example, of 70.1 J/g±0.5 J/g, such as 70.1 J/g±0.25 J/g, or 70.1 J/g±0.1 J/g, where the melting enthalpy is determined by differential scanning calorimetry.

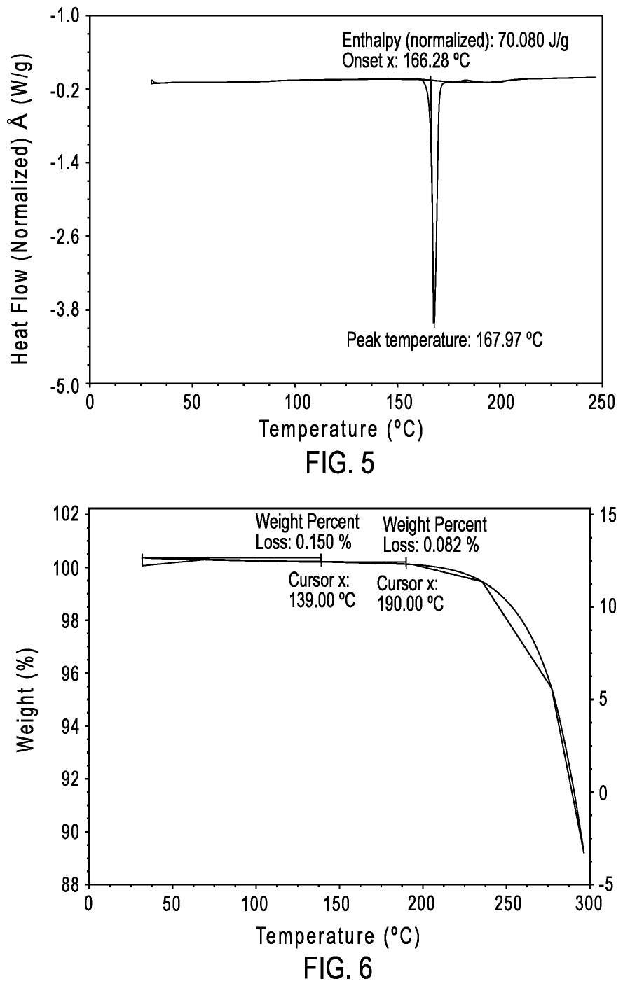

A benzenesulfonic acid cocrystal of compound (1) provided by the present disclosure can exhibit a differential scanning calorimetry curve as substantially shown in FIG. 5.

A benzenesulfonic acid cocrystal of compound (1) provided by the present disclosure can have a weight loss, for example, from 0.1% to 0.3% at a temperature from 180° C. to 200° C., such as from 0.15% to 0.25% at a temperature from 185° C. to 195° C., or from 0.175% to 0.225% at a temperature from 188° C. to 192° C., where the weight loss is determined by thermogravimetric analysis.

A benzenesulfonic acid cocrystal of compound (1) provided by the present disclosure can have a weight loss, for example, of 0.2%±0.05% at 190° C.±5° C., such as 0.2%±0.02% at 190° C.±2.5° C., or 0.2%±0.01% at 190° C.±1° C., where the weight loss is determined by thermogravimetric analysis.

A benzenesulfonic acid cocrystal of compound (1) provided by the present disclosure can exhibit a differential thermal calorimetry curve as substantially shown in FIG. 6.

A benzenesulfonic acid cocrystal of compound (1) provided by the present disclosure can have a columnar morphology and a particle size distribution (D50) from 10 μm to 50 μm as determined using scanning electron microscopy (SEM), where the cocrystal is prepared according to the method described in Example 2.

A benzenesulfonic acid cocrystal of compound (1) provided by the present disclosure can have a columnar morphology and a particle size distribution (D50) from 10 μm to 40 μm as determined using polarized light microscopy (PLM) , where the cocrystal is prepared according to the method described in Example 2.

A benzenesulfonic acid cocrystal of compound (1) provided by the present disclosure can adsorb, for example, from 1.2 wt % to 3.2 wt % water at 25° C./80% RH, such as from 1.4 wt % to 3.0 wt % water at 25° C./80% RH, from 1.6 wt % to 2.8 wt %, from 1.8 wt % to 2.6 wt %, or from 2.0 wt % to 2.4 wt %, where water adsorption is determined by differential vapor desorption.

A benzenesulfonic acid cocrystal of compound (1) provided by the present disclosure can adsorb, for example, 2.2 wt %±0.5% water at 25° C./80% RH, where water adsorption is determined by differential vapor desorption.

A benzenesulfonic acid cocrystal of compound (1) provided by the present disclosure can adsorb, for example, from 55 wt % to 75 wt % water at 25° C./95% RH, such as from 57 wt % to 73 wt % water, from 59 wt % to 71 wt %, from 61 wt % to 69 wt %, or from 63 wt % to 67 wt % water at 25° C./95% RH, where water adsorption is determined by differential vapor desorption.

A benzenesulfonic acid cocrystal of Compound (1) provided by the present disclosure can adsorb 65 wt %±5 wt % water at 25° C./95% RH, where water adsorption is determined by differential vapor desorption.

As described in Example 6, the benzene sulfonic acid cocrystal exhibit a higher solubility than the free form in several biologically relevant media.

A benzenesulfonic acid cocrystal of compound (1) provided by the present disclosure is stable, for example, in an open vial at 25° C./60% RH for 2 weeks or in a closed vial at 60° C. or 1.2M flux-hours for two weeks, and began to show a form chance in an open vial at 40° C./75% RH by 2 weeks.

A para-toluenesulfonic acid cocrystal of compound (1) can comprise, for example, from 0.8 equivalents to 1.2 equivalents, such as from 0.85 equivalents to 1.15 equivalents, from 0.9 equivalents to 1.1 equivalents, or from 0.95 equivalents to 1.05 equivalents of para-toluenesulfonic acid, wherein the stoichiometry is determined by ¹H-NMR.

A para-toluenesulfonic acid cocrystal of compound (1) can comprise 1.0 equivalents of para-toluenesulfonic acid, wherein the stoichiometry is determined by ¹H-NMR.

A para-toluenesulfonic acid cocrystal provided by the present disclosure can be characterized by an XRPD pattern comprising characteristic diffraction peaks at 7.6°±0.2°, 25.0°±0.2°, and 26.7°±0.2° expressed as 2θ angles and determined using Cu-Kα radiation.

A para-toluenesulfonic acid cocrystal provided by the present disclosure can be characterized by an XRPD pattern comprising characteristic diffraction peaks at 7.6°±0.1°, 25.0°±0.1°, and 26.7°±0.1° expressed as 2θ angles and determined using Cu-Kα radiation.

A para-toluenesulfonic acid cocrystal provided by the present disclosure can be characterized by an XRPD pattern comprising characteristic diffraction peaks at 7.6°±0.2°, 8.6°±0.2°, 16.1°±0.2°, 21.0°±0.2°, 25.0°±0.2°, and 26.7°±0.2° expressed as 2θ angles and determined using Cu-Kα radiation.

A para-toluenesulfonic acid cocrystal provided by the present disclosure can be characterized by an XRPD pattern comprising characteristic diffraction peaks at 7.6°±0.1°, 8.6°±0.1°, 16.1°±0.1°, 21.0°±0.1°, 25.0°±0.1°, and 26.7°±0.1° expressed as 2θ angles and determined using Cu-Kα radiation.

A para-toluenesulfonic acid cocrystal provided by the present disclosure can be characterized by an XRPD pattern comprising characteristic diffraction peaks at 7.6°±0.2°, 8.6°±0.2°, 13.4°±0.2°, 16.1°±0.2°, 17.7°±0.2°, 19.3°±0.2°, 20.3°±0.2°, 21.0°±0.2°, 24.9°±0.2°, 25.0°±0.2°, and 26.7°±0.2° expressed as 2θ angles and determined using Cu-Kα radiation.

A para-toluenesulfonic acid cocrystal provided by the present disclosure can be characterized by an XRPD pattern comprising characteristic diffraction peaks at 7.6°±0.1°, 8.6°±0.1°, 13.4°±0.1°, 16.1°±0.1°, 17.7°±0.1°, 19.3°±0.1°, 20.3°±0.1°, 21.0°±0.1°, 24.9°±0.1°, 25.0°±0.1°, and 26.7°±0.1° expressed as 2θ angles and determined using Cu-Kα radiation.

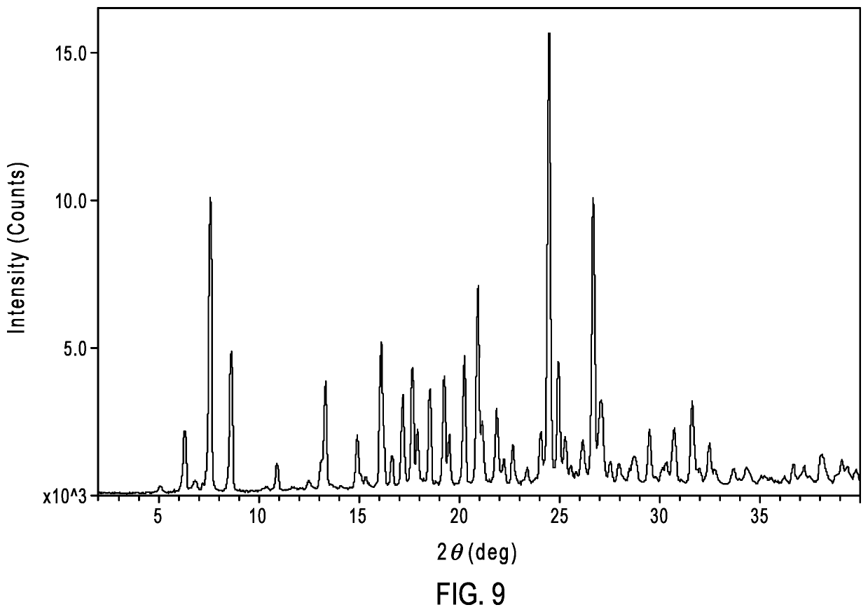

A para-toluenesulfonic acid cocrystal of compound (1) provided by the present disclosure can be characterized by an XRPD pattern as substantially shown in FIG. 9.

A para-toluenesulfonic acid cocrystal of compound (1) provided by the present disclosure can have a melting onset temperature, for example, from 207° C. to 217° C., such as from 208° C. to 216° C., from 209° C. to 215° C., from 210° C. to 214° C., or from 211° C. to 213° C., where the melting onset temperature is determined by differential scanning calorimetry.

A para-toluenesulfonic acid cocrystal of compound (1) provided by the present disclosure can have a melting onset temperature, for example, of 211.6° C.±0.5° C., such as 211.6° C.±0.25° C., or 211.6° C.±0.1° C., where the melting onset temperature is determined by differential scanning calorimetry.

A para-toluenesulfonic acid cocrystal of compound (1) provided by the present disclosure can have a melting enthalpy, for example, from 83 J/g to 93 J/g, from 84 J/g to 92 J/g, from 85 J/g to 91 J/g, from 86 J/g to 90, or from 87 J/g to 89 J/g, where the melting enthalpy is determined by differential scanning calorimetry.

A p-toluenesulfonic acid cocrystal of compound (1) provided by the present disclosure can have a melting enthalpy of 87.7 J/g±0.5 J/g, such as 87.7 J/g±0.25 J/g, or 87.7 J/g±0.1 J/g, where the melting enthalpy is determined by differential scanning calorimetry.

A para-toluenesulfonic acid cocrystal of compound (1) provided by the present disclosure can have a weight loss of from 0.1% to 0.5% at a temperature from 175° C. to 185° C., such as from 0.15% to 0.45% at a temperature from 177° C. to 183° C., or from 0.2% to 0.4% at a temperature from 178° C. to 182° C., where the weight loss is determined by thermogravimetric analysis.

A para-toluenesulfonic acid cocrystal of compound (1) provided by the present disclosure can have a weight loss of 0.3%±0.05% at 180° C.±5° C., such as 0.3%±0.025% at 180° C.±2° C., or 0.3%±0.01% at 180° C.±1° C., where the weight loss is determined by thermogravimetric analysis.

A para-toluenesulfonic acid cocrystal of compound (1) provided by the present disclosure can have a columnar morphology and a particle size distribution (D50) from 5 μm to 30 μm as determined using scanning electron microscopy (SEM), where the cocrystal is prepared according to the method described in Example 1.

A para-toluenesulfonic acid cocrystal of compound (1) provided by the present disclosure can have a columnar morphology and a particle size distribution (D50) from 10 μm to 20 μm as determined using polarized light microscopy (PLM), where the cocrystal is prepared according to the method described in Example 1.

A para-toluenesulfonic acid cocrystal of compound (1) provided by the present disclosure can adsorb, for example, from 0.6 wt % to 1.6 wt % water at 25° C./80% RH, such as from 0.7 wt % to 1.5 wt % water at 25° C./80% RH, from 0.8 wt % to 1.4 wt %, from 0.9 wt % to 1.3 wt %, or from 1.0 wt % to 1.2 wt % water, where water adsorption is determined by differential vapor desorption.

A para-toluenesulfonic acid cocrystal of compound (1) provided by the present disclosure can adsorb, for example, 1.1 wt %±0.3 wt % water at 25° C./80% RH, such as 1.1 wt %±0.2 wt % water at 25° C./80% RH, or 1.1 wt %±0.1 wt % water at 25° C./80% RH, where water adsorption is determined by differential vapor desorption.

A para-toluenesulfonic acid cocrystal of compound (1) provided by the present disclosure can adsorb, for example, from 0.6 wt % to 1.6 wt % water at 25° C./95% RH, such as from 0.7 wt % to 1.5 wt % water at 25° C./95% RH, from 0.8 wt % to 1.4 wt %, from 0.9 wt % to 1.3 wt %, or from 1.0 wt % to 1.2 wt % water, where water adsorption is determined by differential vapor desorption.

A para-toluenesulfonic acid cocrystal of compound (1) provided by the present disclosure can adsorb 1.1 wt %±0.3 wt % water at 25° C./95% RH, such as 1.1 wt %±0.2 wt % water at 25° C./95% RH, or 1.1 wt %±0.1 wt % water at 25° C./95% RH, where water adsorption is determined by differential vapor desorption.

As described in Example 3, the para-toluenesulfonic acid cocrystal exhibit a higher solubility than the free form in several biologically relevant media.

A para-toluenesulfonic acid cocrystal of Compound (1) provided by the present disclosure is stable, for example, in an open vial at 25° C./60% RH for 2 weeks, in an open vial at 40° C./75% RH for 2 weeks, and in a closed vial at 60° C. or 1.2M lux-hours for 2 weeks.

A fumaric acid cocrystal of compound (1) can comprise, for example, from 0.3 equivalents to 0.7 equivalents, such as from 0.35 equivalents to 0.65 equivalents, from 0.4 equivalents to 0.65 equivalents, or from 0.45 equivalents to 0.6 equivalents fumaric acid.

A fumaric acid cocrystal of compound (1) can comprise 0.5 equivalents of fumaric acid.

A fumaric acid cocrystal of compound (1) provided by the present disclosure can be characterized by an XRPD pattern comprising characteristic diffraction peaks at 76.3°±0.2°, 8.0°±0.2°, 11.9°±0.2°, and 25.7°±0.2° expressed as 2θ angles using Cu-Kα radiation.

A fumaric acid cocrystal of compound (1) provided by the present disclosure can be characterized by an XRPD pattern comprising characteristic diffraction peaks at 6.3°±0.1°, 8.0°±0.1°, 11.9°±0.1°, and 25.7°±0.1° expressed as 2θ angles using Cu-Kα radiation.

A fumaric acid cocrystal of compound (1) provided by the present disclosure can be characterized by an XRPD pattern comprising characteristic diffraction peaks at 6.3°±0.2°, 8.0°±0.2°, 11.9°±0.2°, 13.4°±0.2°, 23.7°±0.2°, and 25.7°±0.2° expressed as 2θ angles using Cu-Kα radiation.

A fumaric acid cocrystal of compound (1) provided by the present disclosure can be characterized by an XRPD pattern comprising characteristic diffraction peaks at 6.3°±0.1°, 8.0°±0.1°, 11.9°±0.1°, 13.4°±0.1°, 23.7°±0.1°, and 25.7°±0.1° expressed as 2θ angles using Cu-Kα radiation.

A fumaric acid cocrystal of compound (1) provided by the present disclosure can be characterized by an XRPD pattern comprising characteristic diffraction peaks at 6.3°±0.2°, 8.0°±0.2°, 8.2°±0.2°, 11.9°±0.2°, 13.4°, 16.0°±0.2°, 16.3°±0.2°, 19.7°±0.2°, 20.1°±0.2°, 21.2°±0.2°, 23.7°±0.2°, and 25.7°±0.2° expressed as 2θ angles using Cu-Kα radiation.

A fumaric acid cocrystal of compound (1) provided by the present disclosure can be characterized by an XRPD pattern comprising characteristic diffraction peaks at 6.3°±0.1°, 8.0°±0.1°, 8.2°±0.1°, 11.9°±0.1°, 13.4°, 16.0°±0.1°, 16.3°±0.1°, 19.7°±0.1°, 20.1°±0.1°, 21.2°±0.1°, 23.7°±0.1°, and 25.7°±0.1° expressed as 2θ angles using Cu-Kα radiation.

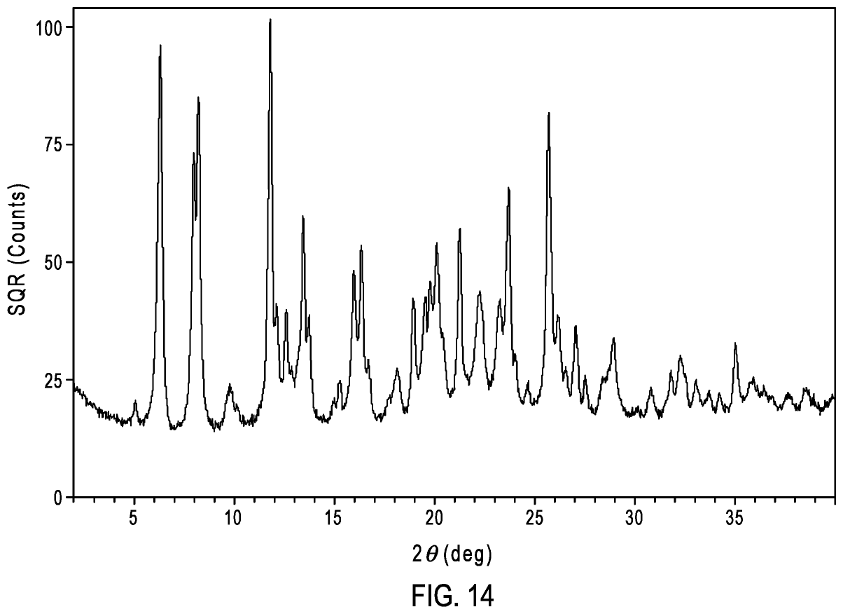

A fumaric acid cocrystal of compound (1) provided by the present disclosure can be characterized by an XRPD pattern as substantially shown in FIG. 14.

A fumaric acid cocrystal of compound (1) provided by the present disclosure decomposes before melting, as determined using differential scanning calorimetry.

A fumaric acid cocrystal of compound (1) provided by the present disclosure can have a weight loss from 0.3% to 0.7% at a temperature from 140° C. to 160° C., such as from 0.35% to 0.65% at a temperature from 145° C. to 155° C., or from 0.4% to 0.6% at a temperature from 147° C. to 153° C., where the weight loss is determined by thermogravimetric analysis.

A fumaric acid cocrystal of compound (1) provided by the present disclosure can have a weight loss of 0.5%±0.1% at 150° C.±5° C., such as 0.5%±0.05% at 150° C.±2.5° C., or 0.5%±0.02% at 150° C.±1° C., where the weight loss is determined by thermogravimetric analysis.

A fumaric acid cocrystal of compound (1) provided by the present disclosure can have a weight loss of from 9% to 17% at a temperature from 240° C. to 260° C., such as from 10% to 16% at a temperature from 245° C. to 255° C., or from 11% to 15% at a temperature from 247° C. to 253° C., where the weight loss is determined by thermogravimetric analysis.

A fumaric acid cocrystal of compound (1) provided by the present disclosure can have a weight loss of 13%±1% at 250° C.±5° C., such as 13%±0.5% at 250° C.±2° C., or 13%±0.5% at 250° C.±1° C. where the weight loss is determined by thermogravimetric analysis.

A fumaric acid cocrystal of Compound (1) provided by the present disclosure can have a fluffy morphology and a particle size distribution (D50) from 10 μm to 30 μm as determined using scanning electron microscopy (SEM), where the cocrystal is prepared according to the method described in Example 4.

A fumaric acid cocrystal of Compound (1) provided by the present disclosure can have a fluffy morphology and a particle size distribution (D50) from 10 μm to 30 μm as determined using polarized light microscopy (PLM), where the cocrystal is prepared according to the method described in Example 4.

A fumaric acid cocrystal of compound (1) provided by the present disclosure can adsorb, for example, from 1.2 wt % to 2.2 wt % water at 25° C./80% RH, such as from 1.3 wt % to 2.1 wt % water at 25° C./80% RH, from 1.4 wt % to 1.9 wt %, from 1.5 wt % to 1.8 wt %, or from 1.6 wt % to 1.7 wt %, where water adsorption is determined by differential vapor desorption.

A fumaric acid cocrystal of compound (1) provided by the present disclosure can adsorb, for example, 1.7 wt %±0.5 wt % water at 25° C./80% RH, such as 1.7 wt %±0.25 wt % water at 25° C./80% RH, or 1.7 wt %±0.1 wt % water at 25° C./80% RH, where water adsorption is determined by differential vapor desorption.

A fumaric acid cocrystal of compound (1) provided by the present disclosure can adsorb, for example, from 2 wt % to 6 wt % water at 25° C./95% RH, such as from 2.5 wt % to 5.5 wt % water, from 3 wt % to 5 wt %, or from 3.5 wt % to 4.5 wt % water at 25° C./95% RH, where water adsorption is determined by differential vapor desorption.

A fumaric acid cocrystal of compound (1) provided by the present disclosure can adsorb, for example, 4.0 wt %±1 wt % water at 25° C./95% RH, such as 4.0 wt %±0.5 wt % water at 25° C./95% RH, or 4.0 wt %±0.2 wt % water at 25° C./95% RH, where water adsorption is determined by differential vapor desorption.

As described in Example 4, the fumaric acid cocrystal exhibit a higher solubility than the free form in several biologically relevant media.

A fumaric acid cocrystal of Compound (1) provided by the present disclosure is stable, for example, in an open vial at 25° C./60% RH for 2 weeks, in an open vial at 40° C./75% RH for 2 weeks, and in a closed vial at 60° C. or 1.2M lux-hours for 2 weeks.

Benzenesulfonic acid, para-toluenesulfonic acid, and fumaric acid cocrystals of compound (1) can be prepared by suspending compound (1) and about 1 equivalents of the acid in acetonitrile. The suspension can be heated, for example, at a temperature from 30° C. to 70° C. such as from 40° C. to 60° C. for from 12 hours to 36 hours such as from 18 hours to 30 hours, and then stirred, for example, at a temperature from 20° C. to 30° C. such as from 23° C. to 27° C. at a rate of about 600 rpm for from 24 hours to 72 hours, such as from 36 hours to 60 hours to provide the corresponding cocrystal of compound (1).

The cocrystal can be dried, for example, at a temperature from 25° C. to 35° C., for from 1 hour to 3 hours.

Pharmaceutical compositions provided by the present disclosure comprise a cocrystal of compound (1) or a combination of cocrystals of compound (1) such as a cocrystal of benzene sulfonic acid and compound (1), a cocrystal of para-toluenesulfonic acid and compound (1) a cocrystal of fumaric acid and compound (1), or a combination of any of the foregoing.

A pharmaceutical composition can comprise a therapeutically effective amount of a cocrystal of compound (1) for treating a disease in a patient.

A pharmaceutical composition can comprise one or more pharmaceutically acceptable carriers, excipients diluents, or combinations of any of the foregoing.

A pharmaceutical composition provided can be formulated for any suitable route of administration including, for example, intradermal, intramuscular, intraperitoneal, intravenous, subcutaneous, intranasal, epidural, oral, peroral, sublingual, intracerebral, intravaginal, transdermal, rectal, inhalation, or topical.

A pharmaceutical composition provided by the present disclosure can be formulated for oral administration.

A pharmaceutical composition formulated for oral administration can comprise any suitable oral dosage form including, for example, tablets, capsules, caplets, sachets, bottles, stick packs, dispersions, and suspensions.

A pharmaceutical composition formulated for oral administration can provide for a modified release profile in the gastrointestinal tract, such as a controlled release profile, a sustained release profile, a pH-release profile, a pulsatile release profile, a timed-release profile, or a delayed release profile. A pharmaceutical composition formulated for oral administration can be configured to release a cocrystal of compound (1) over an intended period of time following ingestion and/or in an intended region of the gastrointestinal tract.

A pharmaceutical composition formulated for oral administration can provide for an immediate release profile.

Thus, cocrystals of compound (1) provided by the present disclosure provide an enhanced oral bioavailability compared to the free from of compound (1).

For example, following oral administration, cocrystals of compound (1) provided by the present disclosure provide a concentration of the cocrystal in a plasma of a patient that is greater than the concentration of compound (1) in the plasma of a patient following oral administration of compound (1).

The cocrystals of compound (1) can exhibit an oral bioavailability, for example, that is greater than 2 times, greater than 5 times, greater than 10 times, or greater than 10 times, the oral bioavailability of the free form of compound (1).

Cocrystals of compound (1) and pharmaceutical compositions comprising a cocrystal of compound (1) can be used to treat a disease in which the etiology of the disease is associated with adenosine A_{2B} receptor activation.

Methods provided by the present disclosure include methods of administering a therapeutically effective amount of a cocrystal of compound (1) or a pharmaceutical composition thereof to a patient to treat cancer, an inflammatory disease, a neurological disease, and other diseases.

A cancer can be a solid tumor or a metastatic cancer.

Examples of cancers include acute lymphoblastic leukemia, acute myeloid leukemia, adrenocortical carcinoma, appendix cancer, astrocytoma, atypical teratoid/rhabdoid tumor, basal cell carcinoma (nonmelanoma), B-cell lymphoma, bladder cancer, bone cancer, brain and spinal cord tumors, brain stem cancer, brain tumor, breast cancer, bronchial tumors, Burkitt lymphoma, carcinoid tumor, carcinoma of head and neck, central nervous system embryonal tumors, cerebellar astrocytoma, cerebral astrocytoma/malignant glioma, cervical cancer, chordoma, chronic lymphocytic leukemia, chronic myelogenous leukemia, colorectal cancer, craniopharyngioma, cutaneous T-cell lymphoma, desmoplastic small round cell tumor, ductal carcinoma, dye cancer, endocrine pancreas tumors (islet cell tumors), endometrial cancer, ependymoblastoma, esophageal cancer, esthesioneuroblastoma, Ewing family of tumors, extracranial germ cell tumor, extrahepatic bile duct cancer, gallbladder cancer, gastric cancer, gastrointestinal carcinoid tumor, gastrointestinal stromal tumor, gestational trophoblastic tumor, glioblastoma, glioma, hairy cell leukemia, head and neck cancer, heart cancer, hematopoetic tumors of the lymphoid lineage, hepatocellular cancer, Hodgkin lymphoma, hypopharyngeal cancer, hypothalamic and visual pathway glioma, IDs-related lymphoma, intraocular melanoma, islet cell tumors, Kaposi sarcoma, kidney cancer, Langerhans cell histiocytosis, laryngeal cancer, leukemia, lip and oral cavity cancer, male breast cancer, malignant fibrous histiocytoma, malignant germ cell tumors, malignant mesothelioma, medulloblastoma, melanoma, Merkel cell carcinoma, mesothelioma, mouth cancer, multiple endocrine neoplasia syndrome, multiple myeloma, mycosis fungoides, myelodysplastic, myeloproliferative neoplasms, nasal cavity and paranasal sinus cancer, nasopharyngeal cancer, neuroblastoma, non-Hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, primary liver cancer, primary metastatic squamous neck cancer with occult, prostate cancer, rectal cancer, renal cell cancer, renal pelvis and ureter, respiratory tract carcinoma, retinoblastoma, rhabdomyosarcoma, salivary gland cancer, sarcoma, Sézary syndrome, skin cancer, small intestine cancer, soft tissue sarcoma, squamous cell carcinoma (nonmelanoma), stomach cancer, supratentorial primitive neuroectodermal tumors, T-cell lymphoma, testicular cancer, throat cancer, thymoma and thymic carcinoma, thyroid cancer, transitional cell cancer, urethral cancer, uterine sarcoma, vaginal cancer, visual pathway and hypothalamic glioma, vulvar cancer, Waldenström macroglobulinemia, Wilms tumor, and systemic and central metastases of any of the foregoing.

In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testis cancer, cervical cancer, vaginal cancer, vulvar cancer, head and neck cancer, or skin cancer. In some embodiments, the cancer is prostate cancer, breast cancer, colon cancer, or lung cancer. In some embodiments, the cancer is breast cancer. In some embodiments, the cancer is a sarcoma, carcinoma, or lymphoma.

Examples of inflammatory diseases include allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

Examples of inflammatory diseases include type 1 hypersensitivity disorders such as chronic obstructive pulmonary disease, asthma, hay fever (allergic rhinitis), atopic eczema, conjunctivitis, angioedema, anaphylaxis, hives, and urticaria.

Examples of other diseases include chronic and acute liver diseases, lung diseases, renal diseases, obesity, cholangitis, wet macular degeneration, sickle cell diseases, post-myocardial infarction such as post-myocardial infarction pericarditis, and heart failure.

A cocrystal of compound (1) provided by the present disclosure or a pharmaceutical composition thereof may be included in a kit that may be used to administer the compound to a patient for therapeutic purposes. A kit can include a pharmaceutical composition comprising a cocrystal of compound (1) suitable for administration to a patient and instructions for administering the pharmaceutical composition to the patient. The kit can be used, for example, for treating cancer or for treating an inflammatory disease. A kit can comprise a cocrystal of compound (1) provided by the present disclosure, a pharmaceutically acceptable vehicle for administering the cocrystal, and instructions for administering the formulation comprising the cocrystal to a patient.

The pharmaceutical compositions can be included in a container, pack, or dispenser together with instructions for administration.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

### ASPECTS OF THE DISCLOSURE

The disclosure is further defined by the following aspects.

Aspect 1. A cocrystal of 3 -ethyl-1-propyl-8-(1-(3 -(trifluoromethyl)benzyl)-1H-pyrazol-4-yl)-3,7-dihydro-1H-purine-2,6-dione and benzene sulfonic acid, para-toluene sulfonic acid, or fumaric acid:

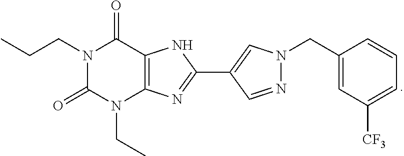 [SMILES: CCCn1c(=O)c2[nH]c(-c3cnn(Cc4cccc(C(F)(F)F)c4)c3)nc2n(CC)c1=O]

Aspect 2. The cocrystal of aspect 1, wherein the cocrystal is a cocrystal of 3-ethyl-1-propyl-8-(1-(3-(trifluoromethyl)benzyl)-1H-pyrazol-4-yl)-3,7-dihydro-1H-purine-2,6-dione and benzenesulfonic acid.

Aspect 3. The cocrystal of aspect 2, wherein the cocrystal of 3-ethyl-1-propyl-8-(1-(3-(trifluoromethyl)benzyl)-1H-pyrazol-4-yl)-3,7-dihydro-1H-purine-2,6-dione and benzenesulfonic acid comprises from 1.0 equivalents to 1.4 equivalents of benzenesulfonic acid.

Aspect 4. The cocrystal of aspect 2, wherein the cocrystal of 3-ethyl-1-propyl-8-(1-(3-(trifluoromethyl)benzyl)-1H-pyrazol-4-yl)-3,7-dihydro-1H-purine-2,6-dione and benzenesulfonic acid comprises 1.2 equivalents of benzenesulfonic acid.

Aspect 5. The cocrystal of any one of aspects 2 to 4, wherein an X-ray powder diffraction pattern of the cocrystal comprises characteristic peaks at 6.8°±0.2°, 17.2°±0.2°, and 23.6°±0.2° expressed as 2θ angles determined using Cu K-α (λ=1.5418 Å) radiation.

Aspect 6. The cocrystal of any one of aspects 2 to 4, wherein an X-ray powder diffraction pattern of the cocrystal comprises characteristic peaks at 6.8°±0.1°, 17.2°±0.1°, and 23.6°±0.1° expressed as 2θ angles determined using Cu K-α (λ=1.5418 Å) radiation.

Aspect 7. The cocrystal of any one of aspects 2 to 4, wherein an X-ray powder diffraction pattern of the cocrystal comprises characteristic peaks at 6.9°±0.2°, 14.7°±0.2°, 15.6°±0.2°, 17.2°±0.2°, 18.8°±0.2°, 22.3°±0.2°, and 23.6°±0.2° expressed as 2θ angles determined using Cu K-α (λ=1.5418 Å) radiation.

Aspect 8. The cocrystal of any one of aspects 2 to 4, wherein an X-ray powder diffraction pattern of the cocrystal comprises characteristic peaks at 6.9°±0.1°, 14.7°±0.1°, 15.6°±0.1°, 17.2°, 18.8°±0.1°, 22.3°±0.1°, and 23.6°±0.1° expressed as 2θ angles determined using Cu K-α (λ=1.5418 Å) radiation.

Aspect 9. The cocrystal of any one of aspects 2 to 8, wherein the cocrystal has a melting onset temperature from 161° C. to 171° C., determined by differential scanning calorimetry.

Aspect 10. The cocrystal of any one of aspects 2 to 8, wherein the cocrystal has a melting onset temperature of 166.3° C.±0.5° C., determined by differential scanning calorimetry.

Aspect 11. The cocrystal of any one of aspects 2 to 10, wherein the cocrystal has a melting enthalpy from 65 J/g to 75 J/g, determined by differential scanning calorimetry.

Aspect 12. The cocrystal of any one of aspects 2 to 10, wherein the cocrystal has a melting enthalpy of 70.1 J/g±0.5 J/g, determined by differential scanning calorimetry.

Aspect 13. The cocrystal of any one of aspects 2 to 12, wherein the cocrystal has a weight loss from 0.1% to 0.3% at a temperature from 180° C. to 200° C., determined by thermogravimetric analysis.

Aspect 14. The cocrystal of any one of aspects 2 to 12, wherein the cocrystal has a weight loss of 0.2%±0.05% at 190° C.±5° C., determined by thermogravimetric analysis.

Aspect 15. The cocrystal of aspect 1, wherein the cocrystal is a cocrystal of 3-ethyl-1-propyl-8-(1-(3-(trifluoromethyl)benzyl)-1H-pyrazol-4-yl)-3,7-dihydro-1H-purine-2,6-dione and para-toluenesulfonic acid.

Aspect 16. The cocrystal of aspect 15, wherein the cocrystal of 3-ethyl-1-propyl-8-(1-(3-(trifluoromethyl)benzyl)-1H-pyrazol-4-yl)-3,7-dihydro-1H-purine-2,6-dione and para-toluenesulfonic acid comprises from 0.8 equivalents to 1.2 equivalents to 1.4 equivalents of para-toluenesulfonic acid.

Aspect 17. The cocrystal of aspect 15, wherein the cocrystal of 3-ethyl-1-propyl-8-(1-(3-(trifluoromethyl)benzyl)-1H-pyrazol-4-yl)-3,7-dihydro-1H-purine-2,6-dione and para-toluenesulfonic acid comprises 1.0 equivalents of para-toluenesulfonic acid.

Aspect 18. The cocrystal of any one of aspects 15 to 17, wherein an X-ray powder diffraction pattern of the cocrystal comprises characteristic peaks at 7.6°±0.2°, 25.0°±0.2°, and 26.7°±0.2° expressed as 2θ angles determined using Cu K-α (λ=1.5418 Å) radiation.

Aspect 19. The cocrystal of any one of aspects 15 to 17, wherein an X-ray powder diffraction pattern of the cocrystal comprises characteristic peaks at 7.6°±0.1°, 25.0°±0.1°, and 26.7°±0.1° expressed as 2θ angles determined using Cu K-α (λ=1.5418 Å) radiation.

Aspect 20. The cocrystal of any one of aspects 15 to 17, wherein an X-ray powder diffraction pattern of the cocrystal comprises characteristic peaks at 7.6°±0.2°, 8.6°±0.2°, 16.1°±0.2°, 21.0°±0.2°, 25.0°±0.2°, and 26.7°±0.2° expressed as 2θ angles determined using Cu K-α (λ=1.5418 Å) radiation.

Aspect 21. The cocrystal of any one of aspects 15 to 17, wherein an X-ray powder diffraction pattern of the cocrystal comprises characteristic peaks at 7.6°±0.1°, 8.6°±0.1°, 16.1°±0.1°, 21.0°±0.1°, 25.0°±0.1°, and 26.7°±0.1° expressed as 2θ angles determined using Cu K-α (λ=1.5418 Å) radiation.

Aspect 22. The cocrystal of any one of aspects 15 to 21, wherein the cocrystal has a melting onset temperature from 207° C. to 217° C., determined by differential scanning calorimetry.

Aspect 23. The cocrystal of any one of aspects 15 to 21, wherein the cocrystal has a melting onset temperature of 211.6° C.±0.5° C., determined by differential scanning calorimetry.

Aspect 24. The cocrystal of any one of aspects 15 to 23, wherein the cocrystal has a melting enthalpy from 83 J/g to 93 J/g, determined by differential scanning calorimetry.

Aspect 25. The cocrystal of any one of aspects 15 to 23, wherein the cocrystal has a melting enthalpy of 87.7 J/g±0.5 J/g, determined by differential scanning calorimetry.

Aspect 26. The cocrystal of any one of aspects 15 to 25, wherein the cocrystal has a weight loss from 0.1% to 0.5% at a temperature from 175° C. to 185° C., determined by thermogravimetric analysis.

Aspect 27. The cocrystal of any one of aspects 15 to 25, wherein the cocrystal has a weight loss of 0.3%±0.05% at 180° C.±5° C., determined by thermogravimetric analysis

Aspect 28. The cocrystal of aspect 1, wherein the cocrystal is a cocrystal of 3-ethyl-1-propyl-8-(1-(3-(trifluoromethyl)benzyl)-1H-pyrazol-4-yl)-3,7-dihydro-1H-purine-2,6-dione and fumaric acid.

Aspect 29. The cocrystal of aspect 28, wherein the cocrystal of 3-ethyl-1-propyl-8-(1-(3-(trifluoromethyl)benzyl)-1H-pyrazol-4-yl)-3,7-dihydro-1H-purine-2,6-dione and fumaric acid comprises from 0.8 equivalents to 0.3 equivalents to 0.7 equivalents of fumaric acid.

Aspect 30. The cocrystal of aspect 28, wherein the cocrystal of 3-ethyl-1-propyl-8-(1-(3-(trifluoromethyl)benzyl)-1H-pyrazol-4-yl)-3,7-dihydro-1H-purine-2,6-dione and fumaric acid comprises 0.5 equivalents of fumaric acid.

Aspect 31. The cocrystal of any one of aspects 28 to 30, wherein an X-ray powder diffraction pattern of the cocrystal comprises characteristic peaks at 76.3°±0.2°, 8.0°±0.2°, 11.9°±0.2°, and 25.7°±0.2° expressed as 2θ angles determined using Cu K-α (λ=1.5418 Å) radiation.

Aspect 32. The cocrystal of any one of aspects 28 to 30, wherein an X-ray powder diffraction pattern of the cocrystal comprises characteristic peaks at 6.3°±0.1°, 8.0°±0.1°, 11.9°±0.1°, and 25.7°±0.1° expressed as 2θ angles determined using Cu K-α (λ=1.5418 Å) radiation.

Aspect 33. The cocrystal of any one of aspects 28 to 30, wherein an X-ray powder diffraction pattern of the cocrystal comprises characteristic peaks at 6.3°±0.2°, 8.0°±0.2°, 11.9°±0.2°, 13.4°±0.2°, 23.7°±0.2°, and 25.7°±0.2° expressed as 2θ angles determined using Cu K-α (λ=1.5418 Å) radiation.

Aspect 34. The cocrystal of any one of aspects 28 to 30, wherein an X-ray powder diffraction pattern of the cocrystal comprises characteristic peaks at 6.3°±0.1°, 8.0°±0.1°, 11.9°±0.1°, 13.4°±0.1°, 23.7°±0.1°, and 25.7°±0.1° expressed as 2θ angles determined using Cu K-α (λ=1.5418 Å) radiation.

Aspect 35. The cocrystal of any one of aspects 28 to 34, wherein the cocrystal has a weight loss from 0.3% to 0.7% at a temperature from 140° C. to 160° C., determined by thermogravimetric analysis.

Aspect 36. The cocrystal of any one of aspects 28 to 34, wherein the cocrystal has a weight loss of 0.5%±0.1% at 150° C.±5° C., determined by thermogravimetric analysis.

Aspect 37. The cocrystal of any one of aspects 28 to 36, wherein the cocrystal has a weight loss from 9% to 17% at a temperature from 240° C. to 260° C., determined by thermogravimetric analysis.

Aspect 38. The cocrystal of any one of aspects 28 to 36, wherein the cocrystal has a weight loss of 13%±1% at 250° C.±5° C., determined by thermogravimetric analysis.

Aspect 39. A method of preparing the cocrystal of aspect 1, comprising: combining 3-ethyl-1-propyl-8-(1-(3-(trifluoromethyl)benzyl)-1H-pyrazol-4-yl)-3,7-dihydro-1H-purine-2,6-dione and an organic in a non-polar solvent to form a suspension, wherein the organic acid is selected from benzenesulfonic acid, para-toluenesulfonic acid, and fumaric acid; heating the suspension; and cooling the heated suspension to a temperature from 20° C. to 30° C. and stirring the cooled suspension; to provide the corresponding cocrystal.

Aspect 40. The method of aspect 39, wherein combining comprises combining the 3-ethyl-1-propyl-8-(1-(3-(trifluoromethyl)benzyl)-1H-pyrazol-4-yl)-3,7-dihydro-1H-purine-2,6-dione with from 0.5 to 1.5 equivalents fumaric acid.

Aspect 41. The method of any one of aspects 39 to 40, wherein the non-polar solvent is acetone.

Aspect 42. The method of any one of aspects 28 to 41, wherein heating the suspense comprises heating at a temperature from 40° C. to 60° C. for from 12 hours to 36 hours.

Aspect 43. The method of any one of aspects 28 to 42, wherein stirring the cooled suspension comprises stirring for from 24 hours to 72 hours.

Aspect 44. A pharmaceutical composition comprising the cocrystal of aspect 1.

Aspect 45. The pharmaceutical composition of aspect 44, wherein the pharmaceutical composition comprises a therapeutically effective amount of the cocrystal for treating a disease in a patient.

Aspect 46. The pharmaceutical composition of aspect 45, wherein the disease is a disease that can be treated by administering an adenosine A_{2B} receptor antagonist.

Aspect 47. The pharmaceutical composition of aspect 45, wherein the disease is cancer.

Aspect 48. The pharmaceutical composition of aspect 45, wherein the disease is an inflammatory disease.

Aspect 49. The pharmaceutical composition of aspect 48, wherein the inflammatory disease is selected from allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

Aspect 50. A method of treating a disease in a patient comprising administering to a patient in need of such treatment a therapeutically effective amount of the cocrystal of aspect 1.

Aspect 51. The method of aspect 50, wherein the disease is a disease that can be treated by administering an adenosine A_{2B} receptor antagonist.

Aspect 52. The method of aspect 50, wherein the disease is cancer.

Aspect 53. The method of aspect 50, wherein the disease is an inflammatory disease.

Aspect 54. The method of aspect 53, wherein the inflammatory disease is selected from allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

Aspect 55. A method of treating a disease in a patient comprising administering to a patient in need of such treatment a therapeutically effective amount of the pharmaceutical composition of aspect 44.

Aspect 56. The method of aspect 55, wherein the disease is a disease that can be treated by administering an adenosine A_{2B} receptor antagonist.

Aspect 57. The method of aspect 55, wherein the disease is cancer.

Aspect 58. The method of aspect 55, wherein the disease is an inflammatory disease.

Aspect 59. The method of aspect 58, wherein the inflammatory disease is selected from allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

Aspect 60. The method of aspect 57, wherein the cancer is selected from bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testes cancer, cervical cancer, vaginal cancer, vulvar cancer, head and neck cancer, and skin cancer.

Aspect 61. The method of aspect 57, wherein the cancer is prostate cancer, breast cancer, colon cancer, or lung cancer.

### EXAMPLES

The following examples describe in detail methods of preparing cocrystals of 3-ethyl-1-propyl-8-(1-(3-(trifluoromethyl)benzyl)-1H-pyrazol-4-yl)-3,7-dihydro-1H-purine-2,6-dione, properties of the cocrystals, and methods of using the cocrystals provided by the present disclosure. It will be apparent to those skilled in the art that many modifications, both to materials and methods, may be practiced without departing from the scope of the disclosure.

### Characterization Instruments and Methods

¹H-NMR was performed using a Bruker Avance-AV® 400M instrument at a frequency of 400 MHZ with a 5 mm PABBO BB-1H/D probe at a temperature of 297.6K and with a relaxation rate of 1 sec.

X-ray powder diffraction (XRPD) was performed using a Bruker D8 Advance® instrument with a Lynxeye® XE T(1D mode) detector at an open angle of 2.94° in continuous scan mode using a Cu/K-α (λ−1.5418 Å) source. The diffraction patterns were obtain over a scan range from 3° to 40° with 0.02° steps, a step duration of 0.12, and a sample rotation speed of 15 rpm.

Differential scanning calorimetry (DSC) was performed using a TA Discovery 2500 or Q2000 instrument over a temperature range from 30° C. to 300° C. at a heating rate of 10° C./min and a nitrogen flow rate of 50 mL/min, using a sample mass from 1 mg to 2 mg.

Thermogravimetric analysis (TGA) was performed using a Discovery 5000 or Q5000 instrument over a temperature range from less than 35° C. to 400° C. at a heating rate of 10° C./min, with a nitrogen flow rate for the balance of 10 mL/min and for the sample of 25 mL/min and using a sample mass of from 2 mg to 10 mg.

Dynamic vapor sorption (DVS) was performed using an Advantage instrument at a total gas flow rate of 200 sccm, an oven temperature of 25° C. with water as the solvent. The humidity cycle was 40-0-95-0-40% RH at step intervals of 10% RH with an equilibration of 0.002 dm/dt (%/min), a maximum, dm/dt stability duration of 60 min, and a maximum dm/dt stage time of 360 min.

Scanning electron microscopy (SEM) was performed using an Phenom® Prox SEM-EDS instrument with a BSD Full detector at magnifications from 200× to 10,000×.

Polarized light microcopy (PLM) was performed using a Nikon LV100POL instrument with a cross polarizer.

### Example 1

### Preparation of Compound (1)

Compound (1) was prepared according to Example 14 of U.S. Application Publication No. 2004/0176399. ¹H NMR (400 MHz, DMSO-d₆): δ 8.56 (s, 1H), 8.14 (s, 1H), 7.71 (br, s, 2H), 7.65-7.55 (m, 2H), 5.53 (s, 2H), 4.05-4.08 (q, J=8.0 Hz, 2H), 3.92-3.80 (m, 2H), 1.58 (sextet, J=8.0 Hz, 2H), 1.25-126 (t, J=8.0 Hz, 3H). 0.88 (t, J=8.0 Hz, 3H) ppm.

The XRPD of Compound (1) is shown in FIG. 1 and exhibits characteristic diffraction peaks at least at 2θ angles of 6.82°, 10.32°, 11.56°, 13.64°, 15.38°, 15.89°, 17.12°, 18.20°, 21.41°, 21.92°, 24.76°, 27.43° as determined using a Cu/K-α (λ=1.5418 Å) source.

The heat flow of the benzenesulfonic acid cocrystal of compound 1 ad determined using differential scanning calorimetry is shown in FIG. 2. The DSC curve exhibits an initial melting onset at 95.14° C., a peak temperature of 104.54° C. and a normalized enthalpy of 3.92 J/g and a second melting onset at 257.2° C., a peak temperature of 258.8° C., and a normalized enthalpy of 96.1 J/g.

A thermogravimetric analysis scan of the benzenesulfonic acid cocrystal of compound (1) is shown in FIG. 3 and exhibits a weight loss of 1.11% at 260° C.

### Example 2

### Benzenesulfonic Acid Cocrystal

The benzenesulfonic acid cocrystal of compound (1) was prepared by weighing 400 mg of compound (1) and 157.5 mg benzenesulfonic acid (˜1.0 equiv.) were into a 40 mL glass vial. Acetonitrile (7.3 mL) was added and the suspension was first heated at 50° C. for 24 hours and then stirred at 25° C. at a rate of 600 rpm for two days. The suspension was then centrifuged to provide the benzenesulfonic acid cocrystal of compound (1). The solids were dried in a vacuum drier at 30° C. for 2 hours. The benzenesulfonic acid cocrystal was obtained as an off-white solid with a yield of 83.1% and a purity of 99.9%. ¹H NMR (400 MHz, DMSO-d₆): δ 8.56 (s, 1H), 8.14 (s, 1H), 7.71 (br. s, 1H), 7.65-7.55 (br. m, 2H+3H from superimposed by excess BSA), 7.35-7.28 (2H from excess BSA), 5.53 (s, 2H), 4.14 (br s, partially superimposed by H₂O from BSA×H₂O), 4.10-4.04 (m, 2H, partially superimposed by H₂O from BSA×H₂O), 3.90-3.80 (m, 2H), 1.65-1.50 (m, 2H), 1.25 (t, J=8.0 Hz, 3H), 0.88 (t, J=8.0 Hz, 3H) ppm.

The XRPD of the benzene sulfonic acid cocrystal of compound (1) is shown in FIG. 1 and exhibits characteristic diffraction peaks at least at 2θ angles of 6.86°, 9.93°, 14.71°, 15.60°, 17.24°, 18.81°, 20.41°, 22.33°, 23.64°, 25.02°, 26.04°, 27.19°, 28.66°, and 31.42° as determined using a Cu/K-α (λ=1.5418 Å) source.

The heat flow of the benzenesulfonic acid cocrystal of compound 1 ad determined using differential scanning calorimetry is shown in FIG. 5. The DSC curve exhibits a melting onset of 166.28° C., a peak temperature of 167.97° C. and a normalized enthalpy of 70.08 J/g.

A thermogravimetric analysis scan of the benzenesulfonic acid cocrystal of compound (1) is shown in FIG. 6 and exhibits a weight loss of 0.15% at 139° C. and a further weight loss of 0.08% at 190° C.

As determined using dynamic vapor sorption (DVS), the benzenesulfonic acid cocrystal is only slightly hygroscopic below 80% RH at 25° C. and then becomes highly hygroscopic, and water absorption reaches 65% at 95% RH. No form change was observed after DVS.

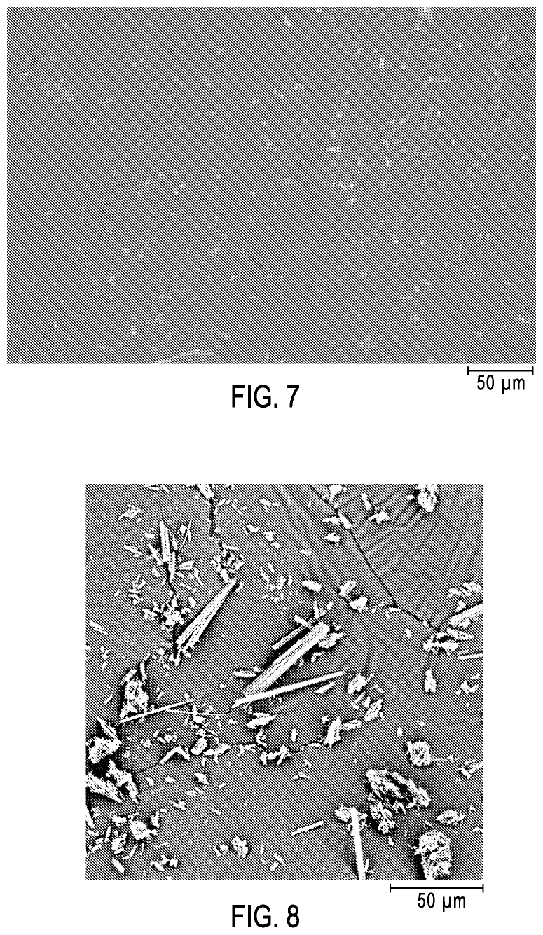

The morphology of the benzenesulfonic acid cocrystals of compound (1) are shown in the PLM and SEM images shown in FIGS. 7 and 8, respectively.

### Example 3

### p-Toluenesulfonic Acid Cocrystal

The p-toluenesulfonic acid cocrystal of Compound (1) was prepared by weighing 400 mg of Compound (1) and 173.1 mg p-toluenesulfonic acid (˜1.0 equiv.) were into a 40 mL glass vial. Acetonitrile (7.3 mL) was added and the suspension was first heated at 50° C. for 24 hours and then stirred at 25° C. at a rate of 600 rpm for two days. The suspension was then centrifuged to provide the p-toluenesulfonic acid cocrystal of Compound (1). The solids were dried in a vacuum drier at 30° C. for 2 hours. The p-toluenesulfonic acid cocrystal was obtained as an off-white solid with a yield of 93.7% and a purity of 99.9%. ¹H NMR (400 MHz, DMSO-d₆): δ 8.64 (s, 1H), 8.21 (s, 1H), 7.78 (br. s, 2H), 7.72-7.66 (m, 2H), 7.55 (d, J=8.0 Hz, 2H, 1.2 eq. p-TSA), 7.19 (d, J=8.0 Hz, 2H, 1.2 eq. p-TSA), 5.60 (s, 2H), 4.55 (br. s, water from p-TSA×H₂O), 4.13 (q, 1H, J=8.0 Hz, 2H), 3.97-3.89 (m, 2H), 2.37 (s, 3H, 1.2 eq. p-TSA), 1.70-1.62 (m, 2H), 1.32 (t, J=8.0 Hz, 3H), 0.95 (t, J=8.0 Hz, 3H) ppm.

The XRPD of the para-toluenesulfonic acid cocrystal of compound (1) is shown in FIG. 9 and exhibits characteristic diffraction peaks at least at 2θ angles of 7.60°, 8.64°, 13.36°, 16.14°, 17.69°, 19.26°, 20.28°, 20.96°, 24.94°, 24.96°, and 26.72° as determined using a Cu/K-α (λ=1.5418 Å) source.

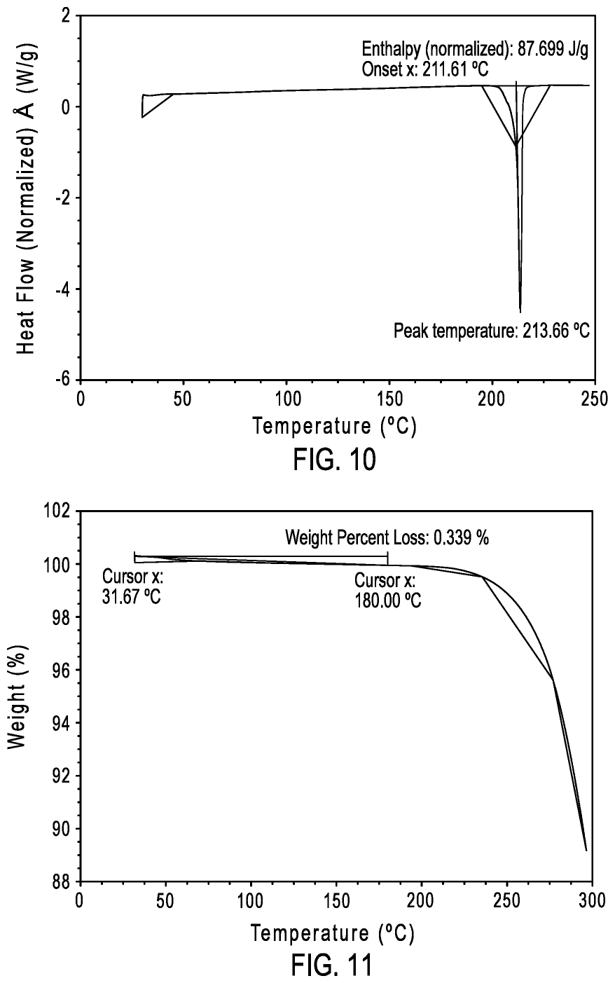

The heat flow of the para-toluenesulfonic acid cocrystal of compound 1 ad determined using differential scanning calorimetry is shown in FIG. 10. The DSC curve exhibits a melting onset of 211.6° C., a peak temperature of 213.7° C. and a normalized enthalpy of 87.7 J/g.

A thermogravimetric analysis scan of the para-toluenesulfonic acid cocrystal of compound (1) is shown in FIG. 11 and exhibits a weight loss of 0.34% at 180° C.

As determined using dynamic vapor sorption (DVS), the para-toluenesulfonic acid cocrystal is only slightly hygroscopic below 80% RH at 25° C. and then becomes slightly hygroscopic, and water absorption reaches 11% at 95% RH. No form change was observed after DVS.

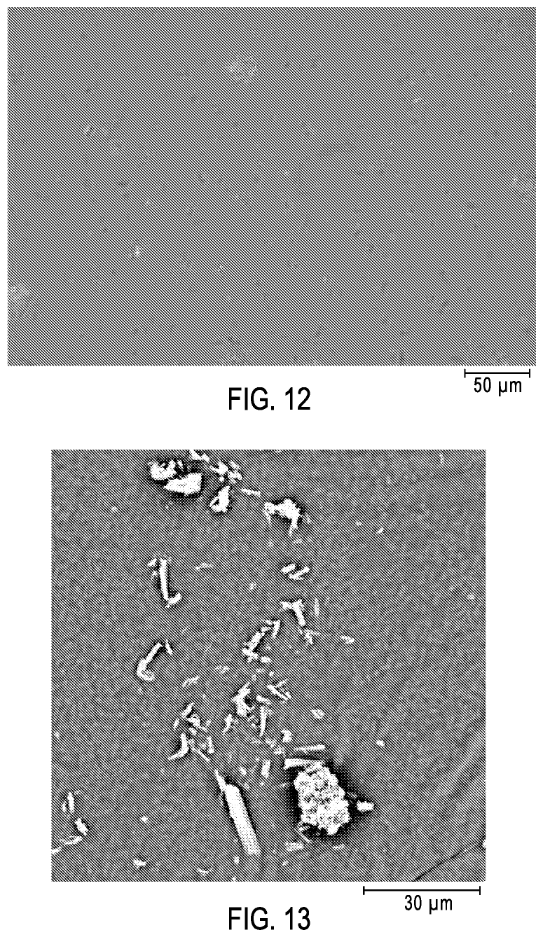

The morphology of the para-toluenesulfonic acid cocrystals of compound (1) are shown in the PLM and SEM images shown in FIGS. 12 and 13, respectively.

### Example 4

### Fumaric Acid Cocrystal

The fumaric acid cocrystal of Compound (1) was prepared by weighing 400 mg of Compound (1) and 110.3 mg fumaric acid (˜1.0 equiv.) were into a 40 mL glass vial. Acetonitrile (7.3 mL) was added and the suspension was first heated at 50° C. for 24 hours and then stirred at 25° C. at a rate of 600 rpm for two days. The suspension was then centrifuged to provide the fumaric acid cocrystal of Compound (1). The solids were dried in a vacuum drier at 30° C. for 2 hours. The fumaric acid cocrystal was obtained as an off-white solid with a yield of 95% and a purity of 99.8%. ¹H NMR (400 MHz, DMSO-d₆): δ 8.55 (s, 1H), 8.13 (s, 1H), 7.70 (br. s, 2H), 7.65-7.57 (m, 2H), 6.63 (s, 1H), 5.53 (s, 2H), 4.06 (q, J=8.0 Hz, 2H), 3.89-3.81 (m, 2H), 1.65-1.53 (m, 2H), 1.25 (t, J=8.0 Hz, 3H), 0.88 (t, J=8.0 Hz, 3H) ppm.

The XRPD of the fumaric acid cocrystal of compound (1) is shown in FIG. 14 and exhibits characteristic diffraction peaks at least at 2θ angles of 6.33°, 8.00°, 8.23°, 11.90°, 13.44°, 15.97°, 16.32°, 19.75°, 20.10°, 21.24°, 23.66°, and 25.66° as determined using a Cu/K-α (λ=1.5418 Å) source.

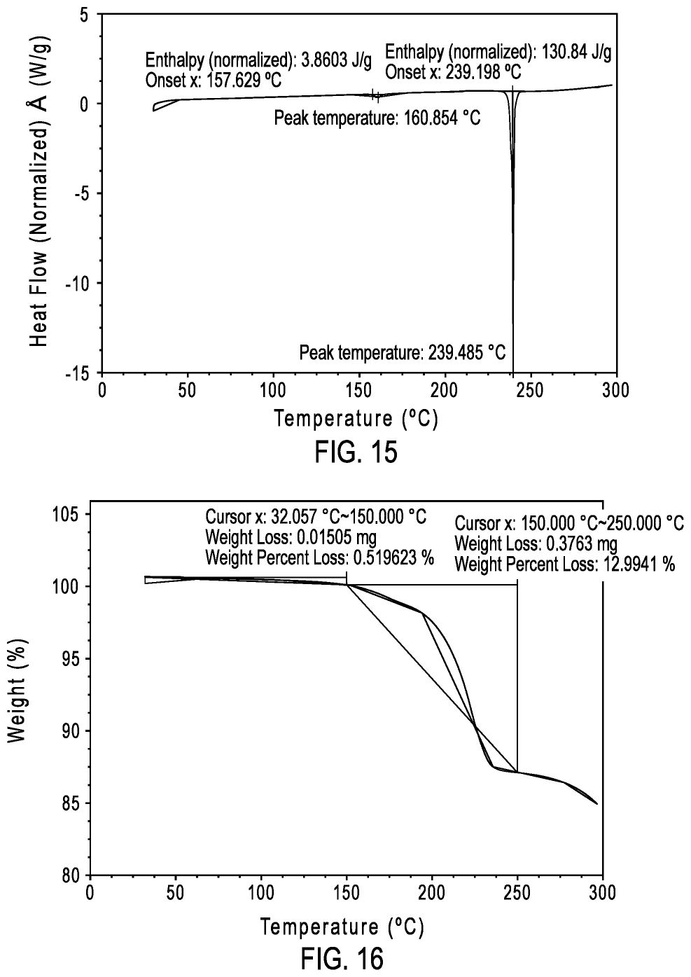

The heat flow of the fumaric acid cocrystal of compound 1 ad determined using differential scanning calorimetry is shown in FIG. 15. The DSC curve exhibits a melting onset of 160.8° C., a peak temperature of 329.5° C. and a normalized enthalpy of 130.8 J/g.

A thermogravimetric analysis scan of the fumaric acid cocrystal of compound (1) is shown in FIG. 16 and exhibits a weight loss of 0.51% at 150° C. and a secondary weight loss of 13.0% at 250 ° C.

As determined using dynamic vapor sorption (DVS), the fumaric acid cocrystal is slightly hygroscopic below 90% RH at 25° C. and then becomes hygroscopic, and water absorption reaches 4% at 95% RH. No form change was observed after DVS.

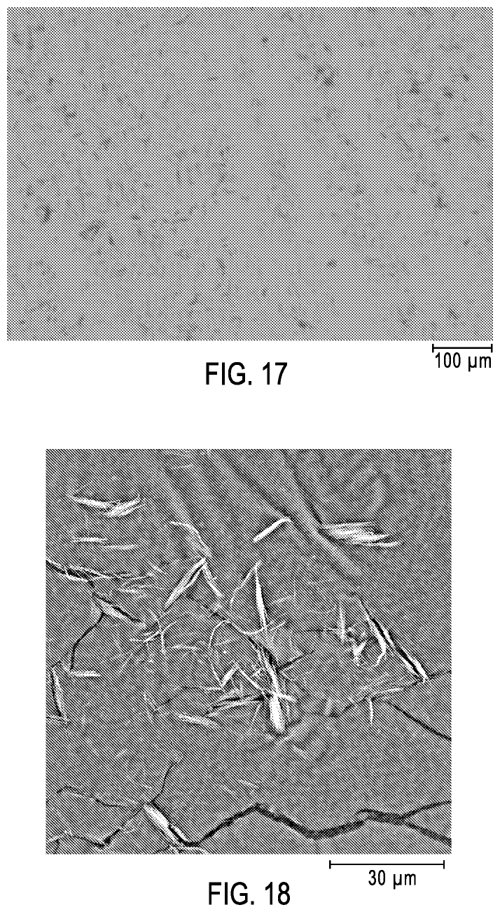

The morphology of the fumaric acid cocrystals of compound (1) are shown in the PLM and SEM images shown in FIGS. 17 and 18, respectively.

### Example 5

### Solubility in Biologically Relevant Fluids

The solubility-time profile of free form compound (1) and the three cocrystals was evaluated in different aqueous media.

The kinetic solubility experiments were conducted for the free form and the three cocrystals in the following media: (1) pH 1.0 (0.1N) HCl solution, (2) pH 4.5 (50 mM) acetate buffer, (3) pH 6.8 (50 mM) phosphate buffer, (4) FeSSIF-v1 (Fed state stimulated intestinal fluid version 1) (pH 5.0), (5) FaSSIF-v1 (Fasted state stimulated intestinal fluid version 1) (pH 6.5), (6) SGF (Simulated gastric fluid) (pH 2.0), and (7) FaSSIF-v1/4% Soluplus®, and (8) SGF/4% Soluplus®.

After combining with the respective media at 37° C., the concentration of the free form or cocrystal and the XRPD was measured at 0.5 hours, 2 hours, and 24 hours.

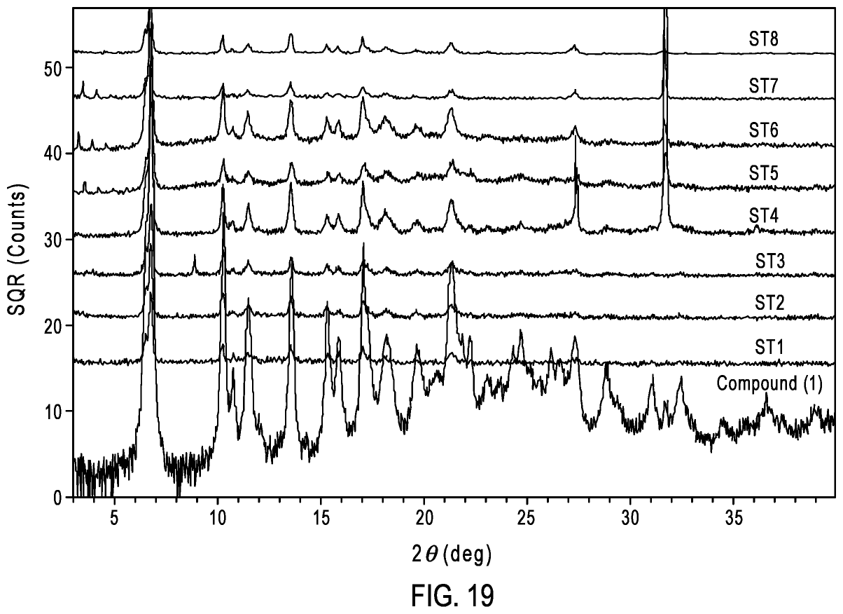

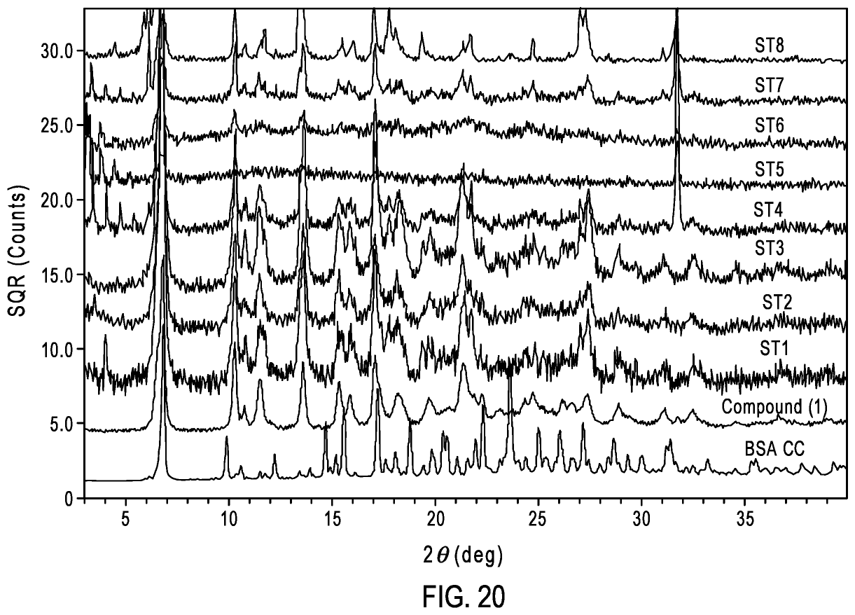

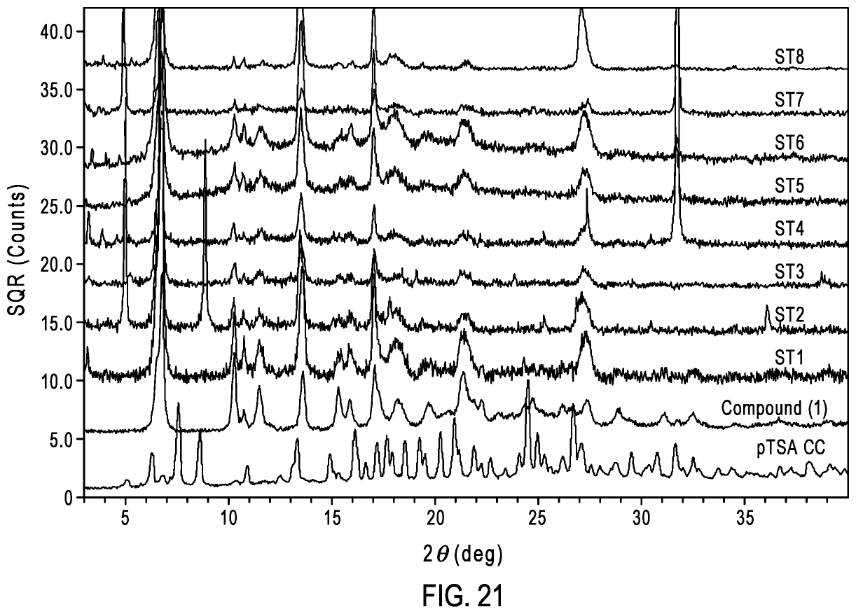

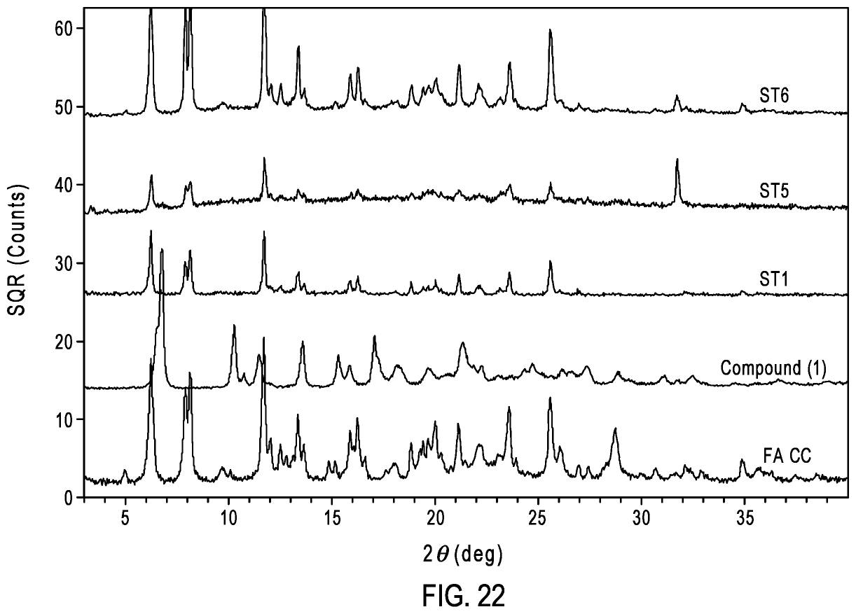

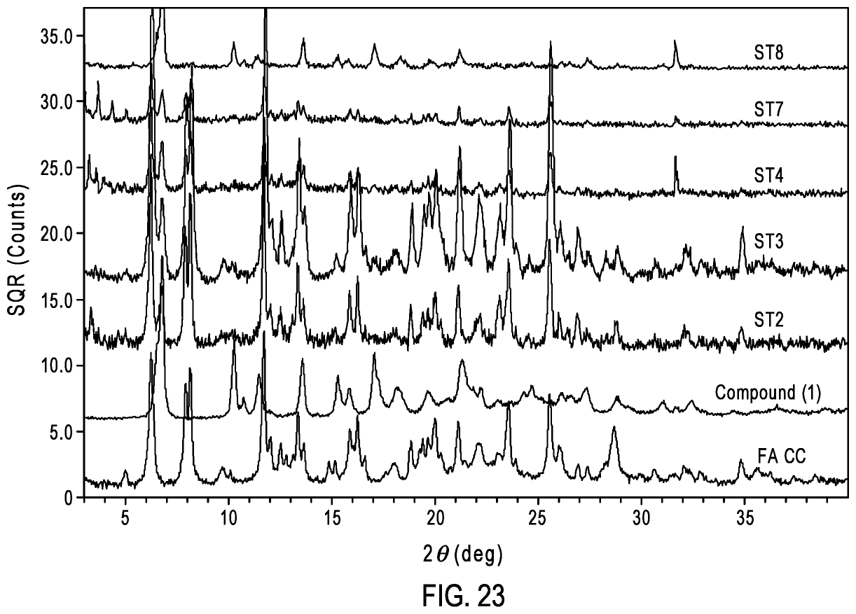

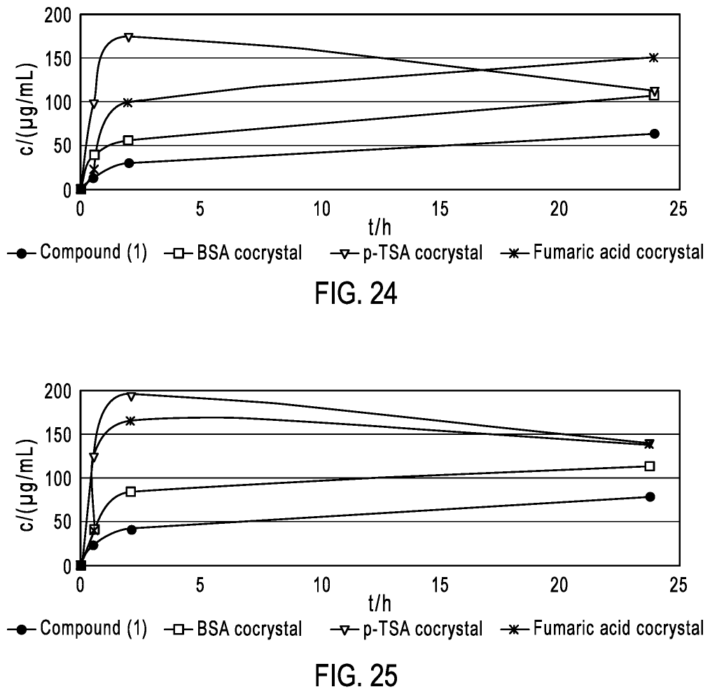

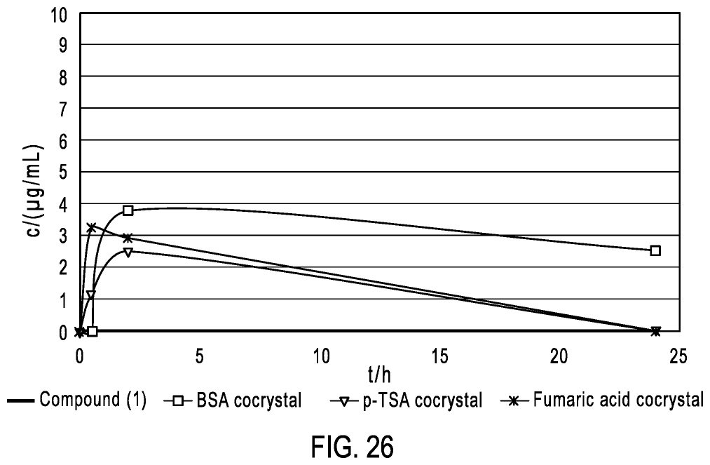

The solubility-time profiles are shown in FIGS. 24-26 and the XRPD patterns are shown in FIGS. 19-23. The XRPD patterns shown in FIGS. 19-23 are associated with the aqueous media described in Table 1 and for the free form (Compound (1), FF), the benzenesulfonic acid cocrystal (BSA CC), the para-toluenesulfonic acid cocrystal (pTSA CC), and the fumaric acid cocrystal (FA CC).

TABLE 1
Aqueous media.

| Test | Aqueous Media |
|---|---|
| ST1 | pH 1.0 (0.1N HCl) |
| ST2 | pH 4.5 (50 mM acetate buffer) |
| ST3 | pH 6.8 (50 mM phosphate buffer) |
| ST4 | FeSSIF-v1 pH 5.0 |
| ST5 | FaSSIF-v1 (pH 6.5) and 4% Soluplus ® |
| ST6 | SGF (pH 2.0) and 4% Soluplus ® |
| ST7 | FaSSIF-v1 (pH 6.5) |
| ST8 | SGF (pH 2.0) |

In certain aqueous media including pH 1.0 HCl solution, pH 4.5 acetate buffer, pH 6.8 phosphate buffer, FeSSIF-v1, and FaSSIF-v1, the solubility of both the free form and the three cocrystals was below the level of quantification (LOQ) at the three different time points (0.5 h, 2 h, and 24 h).

After equilibrating in the aqueous media for 24 hours at 37° C., the free form (Compound (1)) did not show a change in form; the benzenesulfonic acid cocrystal and the para-toluenesulfonic acid cocrystal completely dissociated to the free form; and the fumaric acid cocrystal only partially dissociated to the free form in pH 4.5 acetate buffer, in FeSSIF-v1, and in FaSSIF-v1, and did not show any form change and no amorphous halo in the XRPD pattern in pH 1.0 HCl solution, suggesting that the fumaric acid cocrystal did not dissociate to the free form in the acidic solution.

As shown in FIGS. 24-26, the three cocrystals showed overall higher solubility than that of the free form in SGF, FaSSIF-v1/4% Soluplus®, and SGF/4% Soluplus®, The solubility of the para-toluenesulfonic acid cocrystal first increased and then decreased in FaSSIF-v1/4% Soluplu®s and SGF/4% Soluplus®. The solubility of the fumaric acid cocrystal in SGF/4% Soluplus® also exhibited a similar profile. The solubility of the free form and the benzenesulfonic acid cocrystal increased over time in FaSSIF-v1/4% Soluplus® and SGF/4% Soluplus®. The solubility of the fumaric acid cocrystal in FaSSIF-v1/4% Soluplus® exhibited a similar profile. The solubility of the free base and these three cocrystals maintained in the same magnitude in SGF. After solubility study, the free form showed no form change; the BSA cocrystal and the p-TSA cocrystal dissociated to free form; while the fumaric acid cocrystal converted to a mixture of the cocrystal and amorphous state (free form) in FaSSIF-v1/4% Soluplus® and SGF/4% Soluplus® but dissociated to free form in SGF.

### Example 6

### Bulk Stability

The bulk stability of the free form and the cocrystals was determined by exposing the compounds to conditions of temperature, humidity and/or radiation flux and evaluating the compound by HPLC and XRPD.

Following exposure to 25° C./60% RH in an open vial, 40° C./75% RH in an open vial, 60° C. in a closed vial or 1.2 million lux-hrs for 2 weeks, the free from, the para-toluenesulfonic acid cocrystal, and the fumaric acid cocrystal did not exhibit any significant degradation.

Following exposure to 25° C./60% RH in an open vial, or to 60° C. in a closed vial or 1.2M lux-hrs for 2 weeks, the benzenesulfonic acid cocrystal was stable. At two weeks in an open vial at 40° C./75% RH, the benzenesulfonic acid cocrystal began to show some disassociation.

### Example 7

### Pharmacokinetics of Fumaric Acid Cocrystal in Dogs

The pharmacokinetics of the fumaric acid cocrystal of Example 4 was evaluated following peroral administration to dogs.

Male beagle dogs were separated into two groups of three dogs.

For Group I, the dogs received a single peroral dose of 2 mg/kg (fed) in a dose volume of 5 mL/kg. The dosing formulation consisted of 0.40 mg/mL of the fumaric acid cocrystal in 4% Soluplus® in simulated gastric fluid (SGF) at pH 2.0, as a homogeneous opaque suspension of fine particles.

For Group II, the dogs received a single peroral dose of 10 mg/kg (fed) in a dose volume of 5 mL/kg. The dosing formulation consisted of 2 mg/mL of the fumaric acid cocrystal in 4% Soluplus® in simulated gastric fluid (SGF) at pH 2.0, as a homogeneous opaque suspension of fine particles.

The pharmacokinetic parameters following peroral administration of the fumaric acid cocrystal of Compound (1) is provided in Table 2.

TABLE 2
Pharmacokinetic parameters.

| | AUC_{0-inf} | T_{1/2} | Tₘₐₓ | Cₘₐₓ |
|---|---|---|---|---|
| Dosing Formulation | (ng × h/mL) | (h) | (h) | (ng/mL) |
| 2 mg/kg Cocrystal | 2217 | 14.8 | 4.0 | 148 |
| 10 mg/kg Cocrystal | 21128 | 6.5 | 10.7 | 976 |
| 5 mg/kg Compound (1) | 796¹ | 63.0 | 2.0 | 81 |
| 0.5% methylcellulose | | | | |

¹AUC₀₋₂₄.

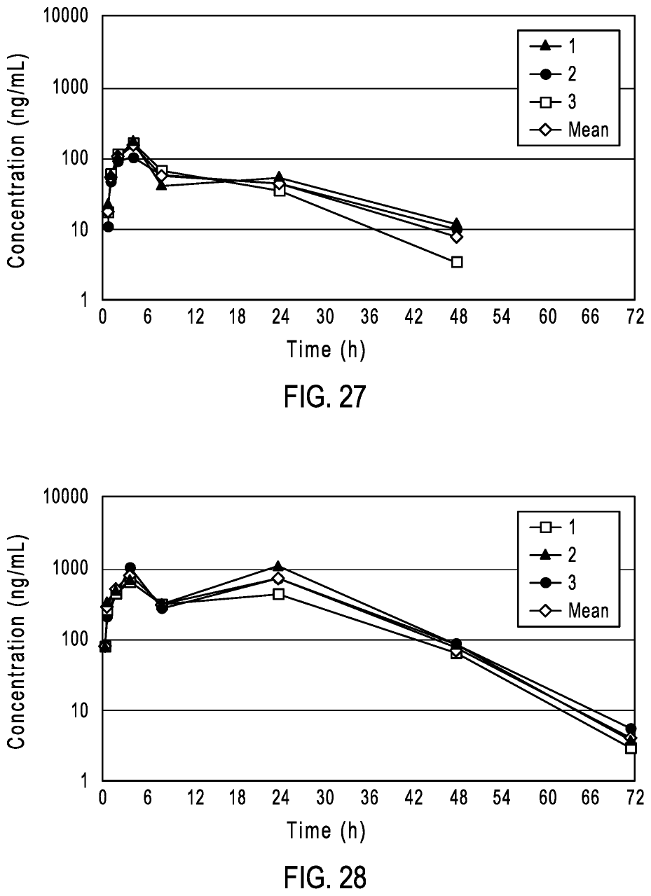

Pharmacokinetic profiles of the concentration of the fumaric acid cocrystal following peroral dosing of either 2 mg/kg or 10 mg/kg to fed dogs is provided in FIGS. 27 and 28, respectively.

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A cocrystal of 3-ethyl-1-propyl-8-(1-(3-(trifluoromethyl)benzyl)-1H-pyrazol-4-yl)-3,7-dihydro-1H-purine-2,6-dione and benzene sulfonic acid, para-toluene sulfonic acid, or fumaric acid:

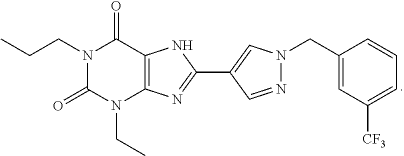 [SMILES: CCCn1c(=O)c2[nH]c(-c3cnn(Cc4cccc(C(F)(F)F)c4)c3)nc2n(CC)c1=O]

2. The cocrystal of claim 1, wherein the cocrystal is a cocrystal of 3-ethyl-1-propyl-8-(1-(3-(trifluoromethyl)benzyl)-1H-pyrazol-4-yl)-3,7-dihydro-1H-purine-2,6-dione and benzenesulfonic acid.

3. The cocrystal of claim 2, wherein the cocrystal of 3-ethyl-1-propyl-8-(1-(3-(trifluoromethyl)benzyl)-1H-pyrazol-4-yl)-3,7-dihydro-1H-purine-2,6-dione and benzenesulfonic acid comprises from 1.0 equivalents to 1.4 equivalents of benzenesulfonic acid.

4. The cocrystal of claim 2, wherein the cocrystal of 3-ethyl-1-propyl-8-(1-(3-(trifluoromethyl)benzyl)-1H-pyrazol-4-yl)-3,7-dihydro-1H-purine-2,6-dione and benzenesulfonic acid comprises 1.2 equivalents of benzenesulfonic acid.

5. The cocrystal of claim 2, wherein an X-ray powder diffraction pattern of the cocrystal comprises characteristic peaks at 6.8°±0.2°, 17.2°±0.2°, and 23.6°±0.2° expressed as 2θ angles determined using Cu K-α (λ=1.5418 Å) radiation.

6. The cocrystal of claim 2, wherein an X-ray powder diffraction pattern of the cocrystal comprises characteristic peaks at 6.8°±0.1°, 17.2°±0.1°, and 23.6°±0.1° expressed as 2θ angles determined using Cu K-α (λ=1.5418 Å) radiation.

7. The cocrystal of claim 2, wherein an X-ray powder diffraction pattern of the cocrystal comprises characteristic peaks at 6.9°±0.2°, 14.7°±0.2°, 15.6°±0.2°, 17.2°±0.2°, 18.8°±0.2°, 22.3°±0.2°, and 23.6°±0.2° expressed as 2θ angles determined using Cu K-α (λ=1.5418 Å) radiation.

8. The cocrystal of claim 2, wherein an X-ray powder diffraction pattern of the cocrystal comprises characteristic peaks at 6.9°±0.1°, 14.7°±0.1°, 15.6°±0.1°, 17.2°, 18.8°±0.1°, 22.3°±0.1°, and 23.6°±0.1° expressed as 2θ angles determined using Cu K-α (λ=1.5418 Å) radiation.

9. The cocrystal of claim 2, wherein the cocrystal has a melting onset temperature from 161° C. to 171° C., determined by differential scanning calorimetry.

10. The cocrystal of claim 2, wherein the cocrystal has a melting onset temperature of 166.3° C.±0.5° C., determined by differential scanning calorimetry.

11. The cocrystal of claim 2, wherein the cocrystal has a melting enthalpy from 65 J/g to 75 J/g, determined by differential scanning calorimetry.

12. The cocrystal of claim 2, wherein the cocrystal has a melting enthalpy of 70.1 J/g±0.5 J/g, determined by differential scanning calorimetry.

13. The cocrystal of claim 2, wherein the cocrystal has a weight loss from 0.1% to 0.3% at a temperature from 180° C. to 200° C., determined by thermogravimetric analysis.

14. The cocrystal of claim 2, wherein the cocrystal has a weight loss of 0.2%±0.05% at 190° C.±5° C., determined by thermogravimetric analysis.

15. The cocrystal of claim 1, wherein the cocrystal is a cocrystal of 3-ethyl-1-propyl-8-(1-(3-(trifluoromethyl)benzyl)-1H-pyrazol-4-yl)-3,7-dihydro-1H-purine-2,6-dione and para-toluenesulfonic acid.

16. The cocrystal of claim 15, wherein the cocrystal of 3-ethyl-1-propyl-8-(1-(3-(trifluoromethyl)benzyl)-1H-pyrazol-4-yl)-3,7-dihydro-1H-purine-2,6-dione and para-toluenesulfonic acid comprises from 0.8 equivalents to 1.2 equivalents to 1.4 equivalents of para-toluenesulfonic acid.

17. The cocrystal of claim 15, wherein the cocrystal of 3-ethyl-1-propyl-8-(1-(3-(trifluoromethyl)benzyl)-1H-pyrazol-4-yl)-3,7-dihydro-1H-purine-2,6-dione and para-toluenesulfonic acid comprises 1.0 equivalents of para-toluenesulfonic acid.

18. The cocrystal of claim 15, wherein an X-ray powder diffraction pattern of the cocrystal comprises characteristic peaks at 7.6°±0.2°, 25.0°±0.2°, and 26.7°±0.2° expressed as 2θ angles determined using Cu K-α(λ=1.5418 Å) radiation.

19. The cocrystal of claim 15, wherein an X-ray powder diffraction pattern of the cocrystal comprises characteristic peaks at 7.6°±0.1°, 25.0°±0.1°, and 26.7°±0.1° expressed as 2θ angles determined using Cu K-α (λ=1.5418 Å) radiation.

20. The cocrystal of claim 15, wherein an X-ray powder diffraction pattern of the cocrystal comprises characteristic peaks at 7.6°±0.2°, 8.6°±0.2°, 16.1°±0.2°, 21.0°±0.2°, 25.0°±0.2°, and 26.7°±0.2° expressed as 2θ angles determined using Cu K-α (λ=1.5418 Å) radiation.

21. The cocrystal of claim 15, wherein an X-ray powder diffraction pattern of the cocrystal comprises characteristic peaks at 7.6°±0.1°, 8.6°±0.1°, 16.1°±0.1°, 21.0°±0.1°, 25.0°±0.1°, and 26.7°±0.1° expressed as 2θ angles determined using Cu K-α (λ=1.5418 Å) radiation.

22. The cocrystal of claim 15, wherein the cocrystal has a melting onset temperature from 207° C. to 217° C., determined by differential scanning calorimetry.

23. The cocrystal of claim 15, wherein the cocrystal has a melting onset temperature of 211.6° C.±0.5° C., determined by differential scanning calorimetry.

24. The cocrystal of claim 15, wherein the cocrystal has a melting enthalpy from 83 J/g to 93 J/g, determined by differential scanning calorimetry.

25. The cocrystal of claim 15, wherein the cocrystal has a melting enthalpy of 87.7 J/g±0.5 J/g, determined by differential scanning calorimetry.

26. The cocrystal of claim 15, wherein the cocrystal has a weight loss from 0.1% to 0.5% at a temperature from 175° C. to 185° C., determined by thermogravimetric analysis.

27. The cocrystal of claim 15, wherein the cocrystal has a weight loss of 0.3%±0.05% at 180° C.±5° C., determined by thermogravimetric analysis

28. The cocrystal of claim 1, wherein the cocrystal is a cocrystal of 3-ethyl-1-propyl-8-(1-(3-(trifluoromethyl)benzyl)-1H-pyrazol-4-yl)-3,7-dihydro-1H-purine-2,6 -dione and fumaric acid.

29. The cocrystal of claim 28, wherein the cocrystal of 3-ethyl-1-propyl-8-(1-(3-(trifluoromethyl)benzyl)-1H-pyrazol-4-yl)-3,7-dihydro-1H-purine-2,6-dione and fumaric acid comprises from 0.8 equivalents to 0.3 equivalents to 0.7 equivalents of fumaric acid.

30. The cocrystal of claim 28, wherein the cocrystal of 3-ethyl-1-propyl-8-(1-(3-(trifluoromethyl)benzyl)-1H-pyrazol-4-yl)-3,7-dihydro-1H-purine-2,6-dione and fumaric acid comprises 0.5 equivalents of fumaric acid.

31. The cocrystal of claim 22, wherein an X-ray powder diffraction pattern of the cocrystal comprises characteristic peaks at 76.3°±0.2°, 8.0°±0.2°, 11.9°±0.2°, and 25.7°±0.2° expressed as 2θ angles determined using Cu K-α (λ=1.5418 Å) radiation.

32. The cocrystal of claim 22, wherein an X-ray powder diffraction pattern of the cocrystal comprises characteristic peaks at 6.3°±0.1°, 8.0°±0.1°, 11.9°±0.1°, and 25.7°±0.1° expressed as 2θ angles determined using Cu K-α (λ=1.5418 Å) radiation.

33. The cocrystal of claim 22, wherein an X-ray powder diffraction pattern of the cocrystal comprises characteristic peaks at 6.3°±0.2°, 8.0°±0.2°, 11.9°±0.2°, 13.4°±0.2°, 23.7°±0.2°, and 25.7°±0.2° expressed as 2θ angles determined using Cu K-α (λ=1.5418 Å) radiation.

34. The cocrystal of claim 22, wherein an X-ray powder diffraction pattern of the cocrystal comprises characteristic peaks at 6.3°±0.1°, 8.0°±0.1°, 11.9°±0.1°, 13.4°±0.1°, 23.7°±0.1°, and 25.7°±0.1° expressed as 2θ angles determined using Cu K-α (λ=1.5418 Å) radiation.

35. The cocrystal of claim 22, wherein the cocrystal has a weight loss from 0.3% to 0.7% at a temperature from 140° C. to 160° C., determined by thermogravimetric analysis.

36. The cocrystal of claim 22, wherein the cocrystal has a weight loss of 0.5%±0.1% at 150° C.±5° C., determined by thermogravimetric analysis.

37. The cocrystal of claim 22, wherein the cocrystal has a weight loss from 9% to 17% at a temperature from 240° C. to 260° C., determined by thermogravimetric analysis.

38. The cocrystal of claim 22, wherein the cocrystal has a weight loss of 13%±1% at 250° C.±5° C., determined by thermogravimetric analysis.

39. A method of preparing the cocrystal of claim 1, comprising:
combining 3-ethyl-1-propyl-8-(1-(3-(trifluoromethyl)benzyl)-1H-pyrazol-4-yl)-3,7-dihydro-1H-purine-2,6-dione and an organic in a non-polar solvent to form a suspension, wherein the organic acid is selected from benzenesulfonic acid, para-toluenesulfonic acid, and fumaric acid;
heating the suspension; and
cooling the heated suspension to a temperature from 20° C. to 30° C. and stirring the cooled suspension;
to provide the corresponding cocrystal.

40. The method of claim 39, wherein combining comprises combining the 3-ethyl-1-propyl-8-(1-(3-(trifluoromethyl)benzyl)-1H-pyrazol-4-yl)-3,7-dihydro-1H-purine-2,6-dione with from 0.5 to 1.5 equivalents fumaric acid.

41. The method of claim 39, wherein the non-polar solvent is acetone.

42. The method of claim 39, wherein heating the suspense comprises heating at a temperature from 40° C. to 60° C. for from 12 hours to 36 hours.

43. The method of claim 39, wherein stirring the cooled suspension comprises stirring for from 24 hours to 72 hours.

44. A pharmaceutical composition comprising the cocrystal of claim 1.

45. The pharmaceutical composition of claim 44, wherein the pharmaceutical composition comprises a therapeutically effective amount of the cocrystal for treating a disease in a patient.

46. The pharmaceutical composition of claim 45, wherein the disease is a disease that can be treated by administering an adenosine A_{2B} receptor antagonist.

47. The pharmaceutical composition of claim 45, wherein the disease is cancer.

48. The pharmaceutical composition of claim 45, wherein the disease is an inflammatory disease.

49. The pharmaceutical composition of claim 48, wherein the inflammatory disease is selected from allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

50. A method of treating a disease in a patient comprising administering to a patient in need of such treatment a therapeutically effective amount of the cocrystal of claim 1.

51. The method of claim 50, wherein the disease is a disease that can be treated by administering an adenosine A_{2B} receptor antagonist.

52. The method of claim 50, wherein the disease is cancer.

53. The method of claim 50, wherein the disease is an inflammatory disease.

54. The method of claim 53, wherein the inflammatory disease is selected from allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

55. A method of treating a disease in a patient comprising administering to a patient in need of such treatment a therapeutically effective amount of the pharmaceutical composition of claim 44.

56. The method of claim 55, wherein the disease is a disease that can be treated by administering an adenosine A_{2B} receptor antagonist.

57. The method of claim 55, wherein the disease is cancer.

58. The method of claim 55, wherein the disease is an inflammatory disease.

59. The method of claim 58, wherein the inflammatory disease is selected from allergy, Alzheimer's disease, anemia such as sickle cell anemia, ankylosing spondylitis, arthritis, atherosclerosis, asthma, autism, arthritis, carpal tunnel syndrome, celiac disease, chronic obstructive pulmonary disease colitis, Crohn's disease, congestive heart failure, dermatitis, diabetes, diverticulitis, eczema, fibromyalgia, fibrosis, gall bladder disease gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

60. The method of claim 57, wherein the cancer is selected from bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testes cancer, cervical cancer, vaginal cancer, vulvar cancer, head and neck cancer, and skin cancer.

61. The method of claim 57, wherein the cancer is prostate cancer, breast cancer, colon cancer, or lung cancer.
